(19) United States
(12) Patent Application Publication
(10) Pub. No.: US 2021/0355428 A1
(43) Pub. Date: Nov. 18, 2021

(54) METHOD FOR DECONTAMINATING A BIOMOLECULE PRODUCTION SYSTEM AND A SYSTEM SUITABLE FOR DECONTAMINATION

(71) Applicant: UNIVERCELLS TECHNOLOGIES S.A., Nivelles (BE)

(72) Inventors: José Castillo, Brussels (BE); Bastien Mairesse, Uccle (BE); Sarah Beck, Wanfercée-baulet (BE)

(21) Appl. No.: 17/286,269

(22) PCT Filed: Oct. 21, 2019
(86) PCT No.: PCT/EP2019/078526

§ 371 Date: Apr. 16, 2021

Related U.S. Application Data
(60) Provisional application No. 62/791,166, filed on Jan. 11, 2019.
(60) Provisional application No. 62/783,464, filed on Dec. 21, 2018.
(60) Provisional application No. 62/767,252, filed on Nov. 14, 2018.
(60) Provisional application No. 62/747,906, filed on Oct. 19, 2018.

(51) Int. Cl. C12M 1/00; C12M 1/34; C12M 3/00; A61L 2/04

(52) U.S. Cl. CPC C12M 47/16; C12M 47/20; C12M 47/12; A61L 2202/21; C12M 41/12; C12M 23/44; A61L 2/04; C12M 47/10

(57) ABSTRACT

The current disclosure concerns a method for decontaminating a virus production system, wherein virus particles are produced in at least one bioreactor and purified from liquids comprising said virus particles thereby generating liquid waste, said method comprising the step of exposing said system or a part thereof to heated air and/or liquid at a temperature of at least 60° C. after viral production took place, thereby eradicating remaining virus particles in said system. In a second and third aspect the current disclosure concerns a virus production system wherein the system comprises a heating device suitable for heating air and/or liquid to a temperature of at least 60° C., and the use of such a system for the production of viruses and/or viral vaccines.

### TECHNICAL FIELD

The invention pertains to the technical field of production of virus derived biotechnological products such as vaccines.

### BACKGROUND

Manufacturing or production of biologics involves, amongst others, the production of proteins such as antibodies, and virus particles and may include the production/synthesis of infectious virus particles as a target biologic i.e. for vaccine production or as a byproduct, i.e. in the production of antibodies. Manufacturing of biologics occurs in isolated environments. During the process of production or purification of the target biomolecule at least one target product stream and at least one waste stream are formed either of which may contain infectious viral products or byproducts. Further, as the production process progresses from upstream to downstream processing, residual infectious virus particles may remain in the system for example on interior surfaces and/or as airborne particles.

Accordingly, there is a need in the art for efficient decontamination of biomolecule production facilities in order to safeguard the safety of the production and purification process.

In addition, due to the vast number of diseases caused by pathogenic bacteria and viruses, there remains a large demand in the field to produce antibodies and viruses efficiently in order to isolate and purify viral proteins, to generate vaccines, or to provide infectious viruses for laboratory studies.

A leak of live virus used in a virus production or testing facility can theoretically occur through contaminated equipment, liquid effluents, air emissions, or incorrect virus disposal. Transmission from the facility to the community is most likely to result from either equipment failure or human error.

Of greatest concern is the inadvertent transmission of infectious virus to the community. There is also the possibility that an unexpected emergency could lead to release of infectious materials from the production system. WO2018/087150 describes a system for the production of viruses or virus derived products, comprising a gas decontamination unit that allows circulation of gasses such as vaporized hydrogen peroxide or formaldehyde through the system in order to decontaminate the external surfaces and the air in the system.

Methods of the prior art for decontamination of biomolecule production systems such as virus production systems of WO2018/087150 are indeed often based on formaldehyde gas fumigation, with formaldehyde being a natural gas at room temperature. One issue with formaldehyde is its residue (either paraformaldehyde or methenamine) which remains on the fumigated area and must be cleaned from all work surfaces. Cleaning the residue is usually done by wiping down all surfaces within isolators or other containment systems with an ammonia-based solution, which is difficult to accomplish. In addition, formaldehyde gas generated from its residue is a concern because of its irritating, toxic and carcinogenic properties.

In addition, the traditional methods of producing viruses from cultured cells are tedious and time consuming, rendering the cost of virus production too high.

In order to obtain products suitable for clinical administration, fast, efficient and safe methods of producing virus or viral proteins in cultured cells are needed.

The present disclosure aims to resolve at least some of the problems mentioned above. The present disclosure thereto provides a biomolecule production system which is well suited for virus production, which minimizes the risk of virus contamination and which assures optimal viral yield and quality in a restricted amount of space. Second, it is also the aim to provide a methodology with a limited amount of operational steps that still provides high yield of biomolecule, with a significant reduction of operation expenses (OPEX) and a high level of containment.

### SUMMARY

The present disclosure provides a method for decontaminating a biomolecule production system according to claim 1. More in particular, a method is provided for decontaminating a biomolecule production system, wherein biomolecules are produced in at least one bioreactor and purified from liquids comprising virus particles, thereby generating liquid waste potentially comprising remaining virus particles, said method comprising the step of exposing said system or a part thereof to heated air and/or liquid at a temperature of at least 60° C. after biomolecule production took place, thereby eradicating remaining virus particles in said system.

In a second aspect, the present disclosure provides a biomolecule production system according to claim 25. More in particular, a biomolecule production system is provided comprising at least a production unit comprising a bioreactor, characterized in that said system further comprises a heating device for heating air and/or liquid to a temperature of at least 60° C. and one or more pumps for circulating the heated air and/or liquid through the biomolecule production system or parts thereof.

The present disclosure also provides a biomolecule production system. More in particular, a biomolecule production system is provided comprising at least one cabinet (isolator) and a bioreactor, characterized in that said system further comprises a waste vessel adapted for inactivating liquid waste, wherein the bioreactor and the waste vessel are housed inside the at least one cabinet.

In an embodiment, the present disclosure provides use of a biomolecule production system according to claim 24. More in particular, the disclosure provides the use of a biomolecule production system, as disclosed, for the production of viruses and/or viral vaccines.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

“Biomolecule” refers to any biological material of interest that is produced in a bioreactor. Biomolecules include, for example, viruses, virus-like particles, viral products, viral vectors, targeted delivery vectors, gene delivery tools, proteins such as antibodies, carbohydrates, lipids, nucleic acids, metabolites and peptides.

“Virus” or “virion” refers to an ultramicroscopic (roughly 20 to 300 nm in diameter), metabolically inert, infectious agent that replicates only within the cells of living hosts, mainly bacteria, plants, and animals: composed of an RNA or DNA core, a protein coat, and, in more complex types, a surrounding envelope.

“Decontamination” refers to a process by which an object or material is freed of living organisms including viruses by inactivation or killing thereof, or by other means.

“Inactivation” refers to rendering an organism inert by application of heat, or other means.

“Bioreactor” refers to any device or system that supports a biologically active environment, for example for cultivation of cells or organisms for production of a biological material. This would include cell stacks, roller bottles, shakes, flasks, stirred tank suspension bioreactors, packed-bed bioreactors, high cell density fixed-bed bioreactors, bioreactors which can be operated in batch and/or in perfusion mode as well as disposable and reusable bioreactors, etc.

“Conduit” refers to a channel for conveying a gas or a liquid, for example air, a buffer or a cell culture harvest. A conduit can be branched.

“Tangential flow filtration (TEE)” refers to a method of membrane filtration in which fluid is forced through a space bounded by one or more porous membranes, where molecules small enough to pass through the pores are eliminated in the filtrate or “permeate”, and molecules large enough to be rejected by the pores remain in the “retentate”. The name tangential flow particularly refers to the fact that the direction of fluid flow is roughly parallel to the membrane, as opposed to so-called dead-end filtration where flow is roughly perpendicular to the membrane.

“Buffer” refers to an aqueous formulation comprising a buffering compound and other components required to establish a specified set of conditions to mediate control of a chromatography method.

“Buffering compound” refers to a chemical compound employed for the purpose of stabilizing the pH of an aqueous solution within a specified range. Phosphate is one example of a buffering compound. Other common examples include but are not limited to compounds such as acetate, citrate, borate, MES, Tris, and HEPES, among many others.

“Purification” refers to the substantial reduction of the concentration of one or more target impurities or contaminants relative to the concentration of a target biomolecule.

“Cell culture harvest”, “culture harvest” and “harvest” are used as synonyms and refer to the unclarified cell culture obtained from culturing cells in a bioreactor. The cultured cells or the grown cells also are referred to as host cells.

An “isolator” or “biosafety cabinet” or “cabinet” are used herein as synonyms and refer to a ventilated laboratory workspace for safely working with biological materials. “Isolator” includes enclosed isolators for containment of materials contaminated with (or potentially contaminated with) pathogens, enclosed biosafety cabinets for containment of materials contaminated with (or potentially contaminated with) pathogens and for protection of the product (e.g. purified target biomolecule) from contamination and laminar flow cabinets for protection of the product (e.g. a purified target biomolecule) from contamination. This ventilated workspace for safely working with materials contaminated with (or potentially contaminated with) pathogens requiring a defined biosafety level is usually equipped with high efficiency particulate air (HEPA) filters and may or may not be open-fronted.

A “containment enclosure” refers to a system of containment, usually involving specialized air handling, airlocks, and secure operating procedures, which prevents the escape of a biological material such as a pathogen, for example, into the external environment or into other working areas. A containment enclosure can comprise one or more isolators.

“Fumigation” refers to the process whereby one or more chemicals are applied in the gaseous state to an area for the purpose of decontaminating the area. Fumigation can be performed inside a containment enclosure and/or an isolator to decontaminate the containment and/or isolator and their contents.

“Good manufacturing practices” or “GMP” refers to quality assurance that ensures that products are consistently produced and controlled to the quality standards appropriate to their intended use and as required by the marketing authorization.

“High efficiency particulate air” or “HEPA” filter refers to a filter capable of removing at least 99.97% of all air particles with a mean aerodynamic diameter of 0.3 micrometers, including microorganisms and viral particles.

“Sterilization” refers to a process that destroys and/or removes all classes of microorganisms (including viruses) and their spores.

### BRIEF DESCRIPTION OF FIGURES

FIGS. 1A and 1B show a schematic overview of a production unit of a biomolecule production system according to embodiments of the disclosure.

FIG. 2 shows a schematic overview of a biomolecule production system according to an embodiment of the disclosure.

FIG. 3 shows the decontamination procedure of a biomolecule production system according to an embodiment of the disclosure.

FIG. 4 shows a schematic overview of the liquid flow in a biomolecule production system according to an embodiment of the disclosure.

FIG. 5A, 5A′ and FIG. 5B show a liquid waste vessel according to an embodiment of the disclosure.

FIG. 6 shows the gas flow according to an embodiment of the system comprising three isolators

### DETAILED DESCRIPTION

The present disclosure relates to a method for decontaminating a biomolecule production system, a biomolecule production system suitable for decontamination using such method and the use of such a system for the production of viruses and/or viral vaccines.

In a first aspect, the disclosure provides a method for decontaminating a biomolecule production system, wherein biomolecules are produced in at least one bioreactor and purified from liquids comprising virus particles thereby generating liquid waste, said method comprising the step of exposing said system or a part thereof to heated air and/or liquid at a temperature of at least 60° C., or at least 70° C., after biomolecule production takes place, thereby inactivating remaining virus particles in said system.

Decontamination of a biomolecule production system based on heat inactivation of viruses offers several advantages. First, heat inactivation of viruses does not require expensive nor complicated equipment, thereby allowing a reduction in the production cost of biomolecules when using the method and/or system as disclosed. In addition, heat inactivation is effective on both enveloped and non-enveloped viruses. Examples of enveloped viruses include but are not restricted to influenza, HIV, mumps virus, yellow fever, measles virus (MeV), Varicella zoster (chickenpox virus), hepatitis A, B, C virus (HPC), ebola virus and rabies virus. Examples of non-enveloped viruses include but are not limited to poliovirus and Rotavirus.

Without wishing to be bound by theory, it is thought that when exposed to heated air and/or liquid at a temperature of at least 60° C., at least 70° C., at least 90° C. the virus particles will denature and thereby become inactive, thus resulting in killing of the virus in the system thereby achieving decontamination of the exposed items, in this case, the biomolecule production system.

In contrast to the problems arising from formaldehyde fumigation, heated air does not leave residues on the treated surfaces. Therefore, using heated air to decontaminate a biomolecule production system also avoids the need to manually clean all formaldehyde residues from the system, which could imply additional health risks as well as could result in the introduction of non-viral contaminants into the system. In addition, decontamination by exposure to heated air does not constitute further health risks nor risks for the environment, which is a significant advantage over the use of carcinogenic formaldehyde. The use of heated liquid and/or heated air to decontaminate internal parts of the system is further advantageous as it allows the agent, which in this case would be airborne or liquid borne heat, to decontaminate areas that are difficult to reach.

Depending on the temperature of the heated air and/or liquid used, decontamination of the system will take more time (at a lower temperature) or less time (at a higher temperature). Efficient decontamination is calculated based on the log₁₀ reduction of the initial viral titer, which can be different for different parts of the system as the viral titer depends on the progression of the virus purification process.

The time needed to reduce the titer of a specific virus by one log₁₀ at a certain temperature is known or can otherwise be measured using methods known to the person skilled in the art. Based on this 1 log₁₀ reduction time, the skilled person can calculate the time needed to decontaminate the system. Accordingly, and in a further embodiment the air and/or liquid is heated to the desired temperature and then maintained at that temperature while the system is exposed to it, for the desired amount of time, preferably until the estimated number of virus particles in the system is below a certain threshold, such as less than 10⁻⁶ viral particles/m³. The skilled person is thus able to calculate what the correct time of exposure of the system is, depending on the virus type and the selected temperature.

In the case of the poliovirus, for example, when the initial viral titer corresponds to 2.04×10¹⁰ TCID₅₀/ml (wherein TCID50 corresponds to a 50% Tissue culture Infective Dose), a 16 log₁₀ reduction is predicted to be achieved when decontamination is performed at a temperature of 70° C. for 80 minutes. After this decontamination, the theoretically calculated number of remaining active viral particles is less than 1×10⁻⁶ TCID₅₀/ml. In an embodiment and to further reduce the risk that a viral particle would remain active after decontamination, the time of treatment can be doubled or tripled, thus requiring a heat decontamination treatment of 6 hours at a temperature of 70° C.

Note that the estimated quantity of virus particles is a calculated estimation and since it is expressed in function of the log₁₀ reduction of viral titer, it can never be equal to zero.

In an embodiment, said system is exposed to heated air and/or liquid at a temperature of at most 99° C. Heating above 99° C. does not further render decontamination more efficient, instead, air and/or liquid at a temperature higher than 99° C. could cause damage to the system and should therefore be avoided. In an embodiment, the heated air and/or liquid is circulated through the system to be decontaminated or through parts thereof, preferably through conduits of the system. Circulation of the heated air and/or liquid at the desired temperature through the system allows to accurately and continuously adjust the temperature of the air and/or liquid, which might otherwise cool down when contacted with the system. Inactivating agents used in the prior art, such as formaldehyde, can leave residues (either paraformaldehyde or methenamine in the case of formaldehyde) on the fumigated area, which must be cleaned from all work surfaces. Cleaning the residue is usually done using an ammonia-based solution. In addition, formaldehyde gas generated from its residue is a concern because of its irritating, toxic and carcinogenic properties. One important advantage of the use of heated air and/or liquid is that the inactivating agent, i.e. the heat, does not leave residues in the bioreactor which could affect host cell growth and biomolecule production in a subsequent production round. In an embodiment, when a heated liquid is used for decontamination, not only inactivation of the virus is obtained but also a cleanup of the system after viral production can be aimed at, including cleansing of the bioreactor, other devices of the system and conduits of the system. The circulating heated liquid can thus remove residual contaminants in addition to the resulting inactivated virus particles which potentially remained in the system after the production and purification process was finished. Residual contaminants can be, for example, cell debris as well as non-target biomolecules produced by the cells (i.e. byproducts), solutes, buffers and compounds which were used for growth of cells or virus purification in a previous production and purification cycle. In a preferred embodiment, heated air is recirculated within the production system during thermal decontamination.

In a second aspect, the disclosure provides a biomolecule production system comprising at least a production unit comprising at least one bioreactor, characterized in that said system further comprises a heating device for heating air and/or liquid to a temperature of at least 60° C., preferably at least 70° C., and one or more pumps for circulating the heated air and/or liquid through the biomolecule production system or parts thereof. In a further embodiment, the biomolecule production system comprises at least a production unit as described above and a purification unit comprising one or more biomolecule purification devices.

In one embodiment, contacting the virus particle with the heated air/and or liquid comprises circulation of the heated air and/or liquid through the biomolecule production system or parts thereof. Part of such a system can be, for example, one isolator and its content. Circulation of the heated air and/or liquid can be obtained, in one embodiment, through the implementation of suitable pumps and/or blowers in the system. Suitable devices for obtaining the circulation of heated air and/or liquid in the system include for example, but are not limited to, peristaltic pumps and mass flow controllers (MFC's).

An additional advantage of a method as disclosed herein, where decontamination of the system is based on circulation of heated liquids through the system or through parts thereof, is that the equipment necessary to circulate the liquid through the system is simple and cheap and in most cases already present in the system as for example pumps and pressure sensors on the conduits are in most cases characteristic of a biomolecule production system. The disclosed method for decontamination of a biomolecule production system therefore contributes to lowering the final production cost of viral products by avoiding the need for specialized equipment for successful decontamination of the system.

Blowers, fans and/or pumps may be provided in the system to induce airflow when the method employs circulation of heated air through the system or through part of the system. Preferably, MFC's are used to control air flow throughout the system. Blowers, fans or MFC's that are used in the system to induce or control airflow of heated air through the system can be located within the volume to be decontaminated (e.g. an isolator or a containment enclosure) or outside that volume. Blowers can be provided as compact, inexpensive equipment, allowing for a reduction in the amount of space occupied by the system.

In addition, the system can comprise one or more HEPA (High Efficiency Particulate Air) filters. HEPA filters are usually present in biomolecule production systems as part of the heating, ventilation and air conditioning (HVAC) equipment. In order to guarantee an extremely low level of particulates (such as dust, airborne organisms or vaporized particles) in biomolecule production systems, HVAC equipment is provided with HEPA filters: air flowing into or out of the respective isolators is filtered through one or a series of HEPA filters in order to guarantee the required level of particulates. Preferably, HEPA filters are present in the system such that a Grade D level of particulates is achieved within one or more isolators present in the system, according to the European standards for GMP. The one or more HEPA filters can be located inside and/or outside of the isolator. In an embodiment, the one or more HEPA filters are located near the exit of, but inside, the isolator to contain the virus therein and prevent contamination of the outside environment.

In an embodiment, air to be heated will enter the system from a location remote from the system's location. In a preferred embodiment, said system resides in a containment enclosure and air is entered from outside the enclosure to the inside. Said entered air will be preferably purified, e.g. by means of a HEPA filter. In a more preferred embodiment, said system resides in one or more isolators in one or more containment enclosures and air enters the isolator and/or containment enclosure from outside the enclosure and/or isolator respectively to inside the isolator and/or containment enclosure. Said entered air will be preferably purified, e.g. by means of a HEPA filter. Heaters present in the system will allow heating of the air in the enclosure until the desired temperature is reached. Heated air will then be circulated through the system or parts of said system.

When a heated liquid is used to expose the system or a part thereof to the desired temperature for decontamination, the liquid can be selected from the group consisting of physiological liquids such as water, buffered saline, 2-(N-morpholino) ethanesulfonic acid (MES) based buffer, tris (hydroxymethyl) aminomethane based buffers, acidic liquids, basic liquids and liquids containing detergents.

In an embodiment, the air and/or liquid is heated using a heating device, including but not limited to a device based on radiant heat, infrared radiation, UV radiation, electrical heating and combinations of the foregoing.

In an embodiment, the air and/or liquid is heated using a heating element, preferably an immersed heating element, such as an electrical resistor. Said heating element converts electrical energy into heat through the process of Joule heating. Electric current through the element encounters resistance, resulting in heating of the element. Suitable heating elements to be used in the context of the current invention are metal heating elements such as resistance wire, ceramic or semiconductors, film heaters, polymer PTC heating elements, composite heating elements. In an embodiment, metal heating elements or composite heating elements are used. Metallic resistance heating elements may be wire or ribbon, straight or coiled. The most common classes of materials used include nichrome, kanthal, cupronickel or etched foil. Composite heating elements may comprise tubular (sheated) comprise a fine coil of nichrome (NiCr) resistance heating alloy wire, that is located in a metallic tube and insulated by magnesium oxide powder; screen-printed metal-ceramic tracks deposited on ceramic insulated metal (generally steel) plates or ceramic core elements use a coiled resistance heating alloy wire threaded through one or more cylindrical ceramic segments.

In an embodiment the system thereto comprises a heating element or heating device comprising an electrical resistor. The resistor converts electric energy into thermal energy which is then transferred to the decontaminating air and/or liquid. Suitable heating elements comprising an electrical resistor include metal heating elements, ceramic heating elements and polymer positive temperature coefficient (PTC) heating elements. The biomolecule production system of the disclosure has a small footprint, thereby permitting the decontamination systems and methods disclosed herein. In some embodiments, the footprint of the system is less than about 50 m², 40 m², 30 m², 20 m², 10 m², 5 m², or less. In some embodiments, the footprint of the system is from about 5 m² to 10 m², 5 m² to 20 m², 5 to 30 m², 5 to 40 m², 5 to 50 m². In an example, the footprint is less than 10 m². However, the small scale does provide for substantial production efficiency. For example, a 7 m² vaccine production system can produce at least 0.5 million doses of a viral vaccine per batch, or about 10⁷ doses per year. As a consequence, this autonomous process has a dramatic impact on the economics of biomolecule production by significantly reducing the cost of goods as well as capital expenditures. Heating of the air and/or liquid for decontamination of this system using an electrical resistor avoids the need for complicated or bulky equipment therefore allowing the method to be performed in a relatively compact biomolecule production system.

In order to assure efficient decontamination of all areas of the system, the system or a part thereof may, in an embodiment, be exposed to infrared and/or ultraviolet (UV) radiation. The biomolecule production system may thereto comprise one or more sources of infrared and/or ultraviolet (UV) radiation, positioned on distinct locations of the system. Such locations might for example be locations that are less efficiently heated using heated air and/or liquid, or by heating the liquid waste vessel or the liquid waste vessel's circulation conduit.

Preferably, sources of infrared radiation are positioned at distinct locations of the system, thereby generating heat of a temperature of at least 60° C., preferably at least 70° C., at least 90° C. and preferably at most 99° C. Infrared radiation, travels through air or space until it encounters an absorbing surface, where it is partially converted to heat and partially reflected. This heat directly warms that surface which can be an object of the system or a viral particle, rather than warming the air. Infrared lights, for example, are easy to implement in a production system, are compact and have a fast response time thus contributing to the low production cost of the system and to the fast and efficient decontamination procedure. In a preferred further embodiment, sources of infrared radiation are positioned at locations which are known in the system as ‘cold spots’ (see below). In an embodiment, both heated air and/or heated liquid as described above, and IR and/or UV are used to decontaminate.

The biomolecule production system according to the current disclosure, comprises at least a production unit. In an embodiment, said production unit comprises at least one bioreactor including a chamber suitable for having a liquid comprising cells and viral particles.

In a preferred embodiment, the system comprises a bioreactor as described in WO 2018 178 376, PCT/EP2018/076354, U.S. 62/608,261 and U.S. 62/711,700 which are hereby incorporated by reference.

In an embodiment, the bioreactor in said system is a batch bioreactor. In another embodiment, the bioreactor is a perfusion bioreactor. In a perfusion bioreactor, equivalent volumes of media are simultaneously added to and removed from the bioreactor, while the cells are retained in the bioreactor. This provides a steady source of fresh nutrients and constant removal of cell (waste) products. Perfusion allows to attain much higher cell density and thus a higher volumetric productivity than conventional bioreactors. In addition, the perfusion bioreactor allows for secreted products to be continuously harvested during the process of removing media. Preferably, the bioreactor is a fixed-bed perfusion bioreactor. A fixed-bed configuration allows for a higher cell density growth to be achieved in the system. Said bioreactor easily allows for a cell density of at least 50 million cells/ml to be achieved. Accordingly, the system makes use of a bioreactor which is smaller than conventional bioreactors, without compromising the high density cell culture capabilities of the bioreactor. Therefore, incorporation of a bioreactor as described allows for a reduction in terms of the space required for the system. Owing to the intensification of cell culture using this type of bioreactor the system is thus provided with a high cell density bioreactor that is small enough to be placed in an isolator. In an embodiment the system is equipped with a bioreactor suitable to be operated both in batch mode and in perfusion mode. This can be advantageous as the bioreactor in the system can be adapted to specific steps in the production and purification process e.g. the bioreactor can be operated in batch mode during inoculation, and in perfusion mode during cell growth.

The system's production unit can comprise one or more concentration devices in addition to the bioreactor. These concentration devices serve to concentrate the viral harvest downstream from the bioreactor to reduce the harvest volume for further downstream processing.

In one embodiment, the system's production unit may comprise one or more liquid waste vessels for collecting liquid waste generated during the cell culture process, and/or waste from cell culture and/or infection. In one embodiment, the production unit or a portion thereof is housed is an isolator, containment enclosure or a combination thereof wherein the one or more waste vessels may be housed inside or outside of the same isolator or containment enclosure or a combination thereof as the bioreactor.

Implementation of one or more concentration devices to reduce the volume of liquid in which the target biomolecule resides in the system of the disclosure further reduces the amount of space occupied by the system as it allows for the volume of the viral harvest to be processed in downstream steps to be reduced. Concentration devices suitable for use in the system include for example, but are not limited to, concentration devices based on filtration and/or size exclusion chromatography. Preferably, the concentration device comprises a microfiltration and/or ultrafiltration device or a size exclusion chromatography device, more preferably, the concentration device comprises a tangential flow filtration (TFF) device. Other devices which can be present in the production unit are filter devices, adsorption devices etc. In some embodiments, the concentrator comprises more than one type of concentration device (e.g., tangential flow filter and dead-end filter).

In another or further embodiment, the biomolecule production system comprises at least a production unit and a downstream purification unit which are in fluent connection with each other. Conduits are provided to allow transfer of liquids, such as buffer or viral harvest, or gases such as air, from and to the various units of the system.

The purification unit downstream from said production unit shall comprise at least one virus purification device which allows further purifying of the virus coming from the production unit. To that purpose, the production and purification unit will be in fluent connection. Purification devices include but are not limited to devices based on filtration (such as ultrafiltration and gel filtration), centrifugation, adsorption, chromatography, precipitation, solvent extraction and combinations thereof. In an embodiment the purification unit of the biomolecule production system comprises one or more purification devices such as a chromatography device downstream from the bioreactor. Said purification unit may also be equipped with one or more liquid waste vessels for collecting liquid waste generated during the purification process, and/or waste from cell culture, infection and the concentration process. One or more conduits connecting an outlet of a purification device to an inlet of a liquid waste vessel are thereto provided in the system to transfer liquid waste from one or more purification devices to one or more liquid waste vessels. Liquid waste collected in the vessel will be preferably decontaminated via heating (see below). In an embodiment, said decontamination is done in line, and during the purification process. In another embodiment, said decontamination is performed at the end of the process.

In an embodiment, said purification unit comprises a clarification device. Optionally, said clarification device is present in the production unit. Clarification devices include but are not limited to devices that allow separation of a solid fraction from a soluble fraction based on precipitation or aggregation of the target biomolecules or the solid impurities. Clarification often involves the addition of one or more chemicals to the cell culture harvest as described for example in WO 2018 178 376 and U.S. 62/670,220 which are hereby incorporated by reference in their entirety. Viral harvest clarification can be considered the first step of downstream processing and ensures removal of cell debris and other contaminants from the viral harvest comprising viral particles. In an embodiment said clarification device comprises one or more filters selected from depth filters, filters comprising diatomaceous earth as filter aid, microfilters and functional filters such as anion exchange depth filters. Clarification allows the removal of remaining cell culture impurities such as host cell DNA and protein residues. Accordingly, this setup allows all residual solid impurities to be removed from the product stream therefore assuring the correct functioning of the subsequent purification steps. The implementation of a clarification device as disclosed here in the system allows compact unit operation and reduces the processing time, thus benefiting the overall economics of the biomolecule production process.

In another or further embodiment, said purification unit comprises a chromatography device with an inlet conduit connected to an outlet conduit of the clarification device. The chromatography device allows further purification of the target biomolecule, such as for example a virus. Preferably, the chromatography device comprises a chromatography column or membrane which offers a high binding capacity, capable of processing a large input volume in a limited number of cycles. In a preferred embodiment, the chromatography device comprises a mixed mode chromatography membrane which is suited for continuous mode operation. Multiple columns or membranes may be present.

In addition to a clarification device and/or a purification device, said purification unit can optionally also comprise a device suitable for final inactivation of a purified virus or for suitable formulation thereof, e.g. for pharmaceutical applications. Note that throughout this disclosure ‘inactivation of the purified virus’ is not to be confused with the inactivation of virus particles which potentially remain in the system after production and purification and which are inactivated during decontamination. During the manufacture of vaccines, inactivation of the purified virus may in some cases be required. Such vaccines are labeled as vaccines comprising inactivated purified virus. The vaccine for polio is an example of an inactivated virus. Inactivation of the purified virus can be performed in the purification unit such as for example described in WO 2018 178 375 which is incorporated by reference in its entirety. Alternatively, inactivation of the purified virus can be performed in a separate purified virus inactivation unit which is comprised in the system. In such embodiment, the outlet conduit of the purification unit is thereto connected to an inlet conduit of the purified virus inactivation unit, in order to allow fluent connection of both units.

The devices, vessels, manifolds, tubings, conduits or portions thereof inside the units used for the production of said biomolecules may be disposable or reusable.

In a most preferred embodiment, said system comprises a production unit, a purification unit and an inactivation unit which are in fluent connection with each other. Production and purification of the virus in said biomolecule production system, is preferably performed in a contained environment such as a biosafety cabinet or isolator. Said isolator may be a cleanroom.

The safety is further guaranteed in an embodiment of the system wherein the system is located in a containment enclosure. This containment enclosure is preferably provided with at least one entrance through which users and/or materials enter the containment enclosure and at least one exit through which users and/or materials exit the containment enclosure. The entrance and exit of the containment enclosure can be opened or closed automatically by a process control device which collects, monitors and/or records data on actions performed by the components of the system. Preferably, the process control device will prohibit outside access to said containment enclosure until decontamination of the system has been recorded. Automatic control of exit from and entry into the containment enclosure guarantees opening of the containment enclosure only under safe conditions, i.e. when full decontamination of the system has been recorded. In addition to collecting, monitoring and/or recording data on actions performed by the components of the system, the process control device can be used to perform the biomolecule production and optionally the biomolecule purification processes in an automated fashion thereby reducing the required human intervention, thereby reducing the risk for exposure of the operator to harmful pathogens or viruses which are part of the production system.

In another or further embodiment, the system's units are placed in one or more isolators within the containment enclosure. When production and purification of the virus is performed in one isolator, the system's liquid waste vessel(s) and/or their respective heating devices which are required for decontamination of the liquid waste are located inside the isolator. When production of the virus and purification thereof are performed in different isolators of the system, at least one liquid waste vessel and/or heating device may be present in each isolator, or at least two liquid waste vessels. This might be the case when, for example, the production unit of a system and the purification unit of that system are located in two different isolators. This set up allows to decontaminate each unit without the need to transport, for example, contaminated liquid waste outside the isolator. This further contributes to the reduction of risk of environmental contamination with active virus particles.

The isolators can be interconnected or separated from one another by partitions which may be present between these isolators and which can be set in an open or closed configuration. In an open configuration, access from one isolator to the other is allowed. Access to the isolators is thus regulated via the opening and closing of said partitions. In another embodiment the units are placed in a single isolator and are separated from each other by partitions which can be set in an open and closed configuration, thereby either allowing or blocking access from one isolator to the other. In another or further embodiment, isolators are provided with an entrance and/or exit opening which can be set in an open or closed configuration. In an open configuration, the contents of the isolator can be accessed by the operator, thereby allowing the operator to place and remove equipment and/or single-use consumable items in the respective isolator. Opening and closing of said partitions, entrance and/or exit may also be automatically controlled by the process control device upon input signals generated by the process, such as the termination of a certain task (e.g. decontamination of the system), in order to further assure the safe use of the system.

The partitions, entrance and/or exit which are provided inside the isolator or as a part thereof can be made from a strong material, for example, aluminum, stainless steel, fiber glass or any other suitable material. The partitions, entrance and/or exit can include lift gate type doors, swing doors, shutters or sliding doors, and may optionally comprise transparent materials such as glass or Plexiglass panels.

In one embodiment the isolator comprises walls wherein one or more of the walls comprise insulation. In an embodiment, at least one of the walls, bottom or ceiling of said isolators is insulated. Suitable insulating material could be any material suitable in the art. In an embodiment, said suitable insulating material is glass wool, fiber glass or neoprene. In an embodiment, at least three of the walls of an isolator are insulated, preferably by glass wool or fiber glass. In a possible embodiment, the bottom, both sides walls and back wall are insulated, preferably by glass wool or fiber glass. In another or further embodiment, the bottom and/or ceiling of said isolator is insulated, for instance by neoprene.

In another or further embodiment, said insulator comprises a glass wall, allowing for the user to observe the inside of said insulator and the production process. In an embodiment, an insulation panel or sheet can be placed in front of said glass wall to insulate the latter. Said insulation panel or sheet may be automatically or manually positioned in front of said glass wall, e.g. when production or activities inside the isolator start. Said insulation panel or sheet may be any material suitable in the art, such as glass wool, fiber glass or neoprene. In an embodiment, said insulation material is neoprene.

A suitable access mechanism, for example, a lock and key mechanism, a pass code punch pad, card swipe, transponder reader, finger print scanner, retina scanner, sensors, automatic identification and data capture methods such as radio-frequency identification (RFID), biometrics (like iris and facial recognition system), magnetic stripes, Optical character recognition (OCR), smart cards and voice recognition, or any other access mechanism, can be provided to unlock one or more of the following: the isolators' partitions, entrance and exit as well as the entrance and exit of the containment enclosure.

Preferably, users that enter the system remain outside the isolators or safety cabinets. In an embodiment, the isolators are configured to be clean rooms which may or may not allow entrance of users.

In order to allow sample taking for quality control procedures, the isolated units are, in an embodiment, provided with flexible sleeves through which the user is allowed limited indirect access to the units, whose contents remain isolated from the user.

In another of further embodiment, one or more isolators used in the system are provided with one or more liquid transport ports and/or rapid transfer port/rapid transfer container (RTP/RTC) systems to allow safe transport of liquids and/or solids into and out of the isolators. Liquids that may be transported from outside the isolator to inside the isolators include for example but are not limited to growth media, infection media, cells, buffers, products such as formaldehyde. Liquids which may be transported from inside the isolator to outside the isolator include, but are not limited to, liquid waste after decontamination thereof has been achieved and possible samples taken throughout the production and purification steps. Liquid containing the target biomolecule (also referred to as the product flow) is transported from one isolator to another during the production and/or purification process through conduits.

These further containment and isolation measures allow the system to be used for production and purification of viruses that constitute a high risk for the user. For example, the system is suited for the purification of large quantities of active virus needed for the manufacture of vaccines consisting of inactivated purified virus. For the latter, high level BSL-3 containment is required. Finally, integration of isolators and containment enclosure in combination with efficient decontamination as part of the system, renders compliance with biological safety rules simpler and less costly, reducing the risks of contamination for the environment and the operators.

Once the viral production has finalized, the system will be decontaminated by blowing or pumping heated air and/or liquid through the system or parts thereof via a method as described above. In a preferred embodiment, only the inside of isolators present in the system are required to be submitted to decontamination using heated air and/or liquid according to the method described above. This can be achieved only when no contaminated material leaves the isolators. The system as described herein allows to perform decontamination of liquid waste generated during the production and/or purification process inside the isolator. The presence of RTP/RTC systems on the isolators further allows to take contaminated material out of the isolators and to decontaminate it safely through autoclaving. The current system thereby provides an isolator with built-in decontamination function.

In an embodiment, said heated air and/or liquid is circulated through the production, purification and if present, the inactivation units and through the devices present in said units. This is achieved by pumping the heated air or liquid through the conduits connecting the devices in said units and allowing passage of the heated air and/or liquid through the devices. Alternatively, said heated air and/or liquid is provided to the system, and monitoring of its temperature leads to its replacement by freshly heated air when required (i.e. when the temperature thereof decreases).

In an embodiment, one or more units or isolators may undergo a decontamination cycle as described above, whereas other units or insulators may be in an active (production) mode. For instance, in an embodiment, the production and purification units/isolators may, once they have terminated their activities, be decontaminated, whereas the downstream inactivation unit or isolator is still active and still performing the inactivation cycle. In another embodiment, all units or isolators will be decontaminated simultaneously.

To that purpose, the units or isolators may in an embodiment be provided with independent decontamination units which are able to run a thermal decontamination in the unit as described above, that is enabling the passage of heated air or liquid through the system. In an embodiment, the decontamination unit will be present inside an isolator. In another embodiment, the decontamination unit is positioned outside the isolators. In another embodiment the decontamination unit may be mobile. In another embodiment, the decontamination unit is positioned outside the isolators and is connectable to an isolator of choice. In an embodiment, decontamination of the units or isolators may be sequential, in another embodiment, decontamination of the units or isolators may be simultaneous. In an embodiment, each isolator may be provided with a decontamination unit, allowing simultaneous decontamination if required. Within a biomolecule production system, some areas, which are also referred to as ‘cold spots’ can be less accessible to heat, than others, or might be heated slower than others. It is thus preferable that potentially remaining viral particles in all areas of the system are equally heated to guarantee uniform decontamination.

In a further embodiment, the production system comprises at least one unit adapted for producing or purifying a biologic material comprising or potentially comprising active virus particles (pathogens); and a liquid waste vessel adapted to decontaminate a liquid waste derived from the production system. During production of viral particles for vaccine production, liquid waste is generated. Such liquid waste often comprises viral particles which cannot be released to the environment unless proper inactivation and decontamination takes place. In an embodiment of the current disclosure, the liquid waste generated during the biomolecule production and purification process will be collected in at least one liquid waste vessel. Said liquid waste vessel is equipped to heat said liquid waste. Accordingly, in an embodiment, the biomolecule production system comprises one or more liquid waste vessels suitable for receiving liquid waste. In a preferred embodiment, both the production unit and the purification unit comprise one or more liquid waste vessels which are equipped to heat said liquid waste and thus decontaminate the content. In another embodiment, one or more liquid waste vessels comprise a heating element inside the vessel.

Liquid waste generated in the production unit and more specifically from devices such as the concentrator in the production unit will be collected in one or more liquid waste vessels which are in fluent connection with said devices such as the concentrator. In an embodiment, the flow-through fraction of the concentrator which can be considered waste will be directed to the liquid waste vessels whereas the retentate comprising the product of interest (being the virus) will be transferred to downstream units for further processing.

In another or further embodiment, the one or more purification units of the system will equally be equipped with one or more liquid waste vessels for collecting and decontaminating liquid waste generated during the virus purification process. Liquid waste may be generated from the chromatography step in the purification unit as well as from other devices such as clarification devices. Conduits between the chromatography devices and the liquid waste vessel and/or between the clarification devices and the waste vessel will be provided, allowing liquid waste transport to said vessels.

Decontamination of the collected liquid waste in the vessels occurs via heat inactivation. For this purpose, said liquid waste vessels are equipped to heat said liquid waste to a temperature of at least 60° C., preferably at least 70° C., at least 90° C. and preferably at most 99° C. Heating of the liquid waste to a temperature of at least 60° C., preferably at least 70° C., at least 90° C. and preferably at most 99° C. assures inactivation of virus particles which are potentially present in the liquid waste to attain decontamination of the liquid waste. Decontamination of the liquid waste as part of the system, simplifies the waste disposal procedure as the waste no longer presents a safety hazard once decontaminated and can be easily disposed of.

In an embodiment, the conduits of the system comprise pumps, valves and flow meters or sensors to control and monitor the flow of liquid from, for example, the concentrator to the bioreactor and/or liquid waste vessel. In an embodiment, the system's conduits allowing liquid waste transport to the liquid waste vessel are equipped with check-valves that prevent backflow of the waste liquid from the liquid waste vessel.

In one embodiment, said waste vessel is adapted for heating. The waste vessel may have any shape such as for example, but not limited to a cylindrical shape, a parallelepipedal shape, or a spherical shape. In a preferred embodiment, the waste vessel has a cylindrical shape. This offers the advantage that cylinders are easier to manufacture, have superior mechanical resistance when heating and show less heat exchange with the environment as compared to, for example, a parallelepiped shaped waste vessel. The liquid waste vessel can be made of any material that resists heat. Suitable materials include thermoplastic polymers which are light in weight and possess a high impact strength. In a preferred embodiment, the liquid waste vessel is made of polypropylene homopolymer and polypropylene random copolymers. These materials offer the advantages that they can be assembled using heat fusion welding, they are suitable for working at the desired temperatures, they have a high chemical resistance and they have good thermal insulating properties.

In a further embodiment, the waste vessel is provided with a heating device allowing heating of the vessel and its content to the desired temperature. Useful heating systems for heating the liquid waste include but are not limited to the presence of electrical resistors inside the liquid waste vessel, the provision of heating jackets encasing the liquid waste vessel, the heating of the contaminated liquid waste outside the liquid waste vessel, and combinations thereof.

In an embodiment, heating will occur as soon as possible, once a minimal of liquid is present in said vessel. In another embodiment, heating will occur once the production process or the purification process in the respective unit has been completed. In yet another or further embodiment, heating of the liquid waste in a liquid waste vessel or heating a liquid waste vessel will occur once a maximal amount of liquid is present in said vessel.

In an embodiment, heating of the vessel occurs ultrasonic, UV or IR devices present near, on or in said vessel.

In an embodiment, a heating system for heating the liquid waste inside the liquid waste vessel is provided which comprises an heating element such as an electrical resistor which may be present in said vessel or which is adapted for insertion in said vessel, and which allows heating of the content of said vessel. By preference, the surface area of the heating element will be as large as possible and the heating element will be positioned inside the vessel, near or on the bottom of said vessel, in order to allow optimal heating of its content. An example of a preferred heating element is a wire resistor, preferably a circular wire resistor in a coiled configuration, which is positioned on the bottom of the vessel or near the bottom at the lower level of the vessel. Fixation of the heating element to the vessel may occur via the lateral surface of the vessel, or via the lid of the vessel (top fixation). In the latter configuration, a non-heating connection between the lid and the heating element will be present.

Preferably, heating begins once the heating element is completely immerged in liquid waste. One or more level sensors can thereto be provided in the liquid waste vessel to indicate that the minimal volume of liquid waste required to fully immerge the resistor has been reached. In a further embodiment the heating element can be reversibly or permanently provided inside the liquid waste vessel. When the heating element such as the electrical resistor can be removed from the liquid waste bottle, the element's size and shape can be adapted to fit through an opening of the liquid waste vessel. This allows efficient cleaning of the liquid waste vessel and the resistor prior to the start of a production and purification process.

Alternatively, the liquid waste vessel can be provided as a closed vessel. A heating element such as an electrical resistor and optionally also one or more sensors present in the vessel are put in place before permanently attaching the bottom and/or lid. In addition connections for connecting the required conduits are provided on a surface of the vessel or on a lid of said vessel.

Accordingly, the current disclosure provides a liquid waste vessel with built-in decontamination capacity. This is especially advantageous when single-use of the liquid waste vessel is envisioned and a disposable liquid waste vessel is desirable. In another or further embodiment, an insulation layer is provided to the liquid waste vessel comprising a heating element such as an electrical resistor. In a preferred embodiment, the insulation has a thermal conductivity of at most 0.1 W/mK, preferably at most 0.08 W/mK, more preferably at most 0.06 W/mK, most preferably at most 0.04 W/mK. The insulation layer is preferably provided on the outside of said vessel and allows to limit heat loss during decontamination, wherein a lower thermal conductivity increasingly contributes to improving the efficiency of the decontamination process.

In an embodiment, the liquid waste vessel can comprise one or more sensors. As mentioned above, the liquid waste vessel can be provided with one or more level sensors which allow to monitor liquid level inside the vessel. Preferably, at least one level sensor is present which allows to monitor if the resistor is immerged in liquid.

More preferably, at least one additional level sensor is present which further allows to monitor when the bottle is fully filled. A third and further level sensors could be present which allows detection of a predefined level of volume in the vessel (e.g. 5 L of volume).

Suitable level sensors known in the art are capacitive, ultrasonic, optic or floating switch sensors. By preference, floating switch sensors are being used. The latter work as a magnetic switch that activates when the liquid lifts the float. Either a single multi-float vertical sensor or a multiple side-mounted single float sensor is being used. By preference, side-mounted single float sensors are used.

In addition to one or more level sensors, the liquid waste vessel can comprise one or more temperature sensors that allow monitoring of the temperature of the liquid inside the liquid waste vessel. By preference, at least one temperature sensor is provided inside the liquid waste vessel near the heating element (hot spot). Monitoring the temperature of the liquid near the element allows to avoid an unwanted excess of temperature which could cause gas formation accompanied by an undesired increase in the pressure inside the liquid waste vessel. In another or further embodiment at least one temperature sensor is provided inside the liquid waste vessel at a ‘cold spot’. As already described above, cold spots are areas which are less accessible to heat than others or which are heated slower than others. The provision of a temperature sensor at a cold spot inside the liquid waste vessel allows to ensure efficient decontamination of the entire liquid volume present inside the decontamination vessel during the decontamination process.

In another embodiment, said vessels may be completely or partially provided with a heating jacket at their circumference, allowing heating of the liquid to a desired temperature. Alternatively, the equipment to heat the liquid waste can comprise a circulation conduit, allowing circulation of liquid waste from and to said vessel, and wherein said conduit comprises a heating device such as an electrical resistor for heating said liquid waste during circulation. Accordingly, the conduit is heated during circulation of the liquid waste to a temperature of at least 60° C., more preferably to a temperature of at least 70° C., at least 90° C. and preferably at most 99° C., thereby heating the liquid waste passing through that conduit and inactivating virus particles which might be present in that liquid waste. Heating of the liquid waste in a circulation conduit may allow faster and more accurate heating, as compared to heating of the liquid waste in a liquid waste vessel or heating a liquid waste vessel. In another or further embodiment, chemicals are added to the vessel to supplement inactivation.

In an embodiment, said vessel may contain an inner or outer layer or coating to promote heat conductivity or to provide insulation. In an embodiment, such layer is attached to said vessel by means known in the art such as gluing. Suitable materials to be used may include polymers or metals such as aluminum.

While it will be understood by one skilled in the art that the liquid waste vessels can have any dimension suitable to be used in the above described system, the vessels will have a preferred volume range of between 1 to 100 L, more preferably between 5 and 100 L, even more preferably between 5 and 80 L. In an embodiment, the dimensions of the liquid waste vessels present in the system will be the same. In another embodiment, the dimensions will vary, depending on the location of the waste vessels in the system and its functionality (e.g. collecting and decontaminating of liquid waste from the upstream or downstream process).

For example, a system can be provided with one or more liquid waste vessels in both the production unit or isolator (upstream) and in the purification isolator or unit (downstream). In an embodiment, the size of the liquid waste vessel(s) in the production unit or isolator can be smaller compared to the vessel(s) of the purification unit. In an example, the vessel(s) in the production unit or isolator will have a volume of 10 L, while those present in the purification unit of isolator may be 70 L.

In an embodiment, said waste vessels are single use. In another embodiment, said vessels may be used for multiple runs.

When two or more liquid waste vessels are present in a unit, these vessels may be fluidly connected to each other by conduits. A switch mechanism which might comprise valves may be present on the respective conduits to allow switching liquid waste transfer from one vessel to another. Switching can be performed manually but is preferably performed on an automated fashion based on the measurement of liquid level inside the respective liquid waste vessel using a level sensor which is provided inside the vessel. This allows sequential use of the vessels which can be advantageous especially when decontamination of large waste volumes is required. Accordingly, the presence of two or more liquid waste vessels allows collection and decontamination of large quantities of liquid waste.

In an embodiment, the vessels are filled in parallel. In another embodiment, the vessels are filled sequentially, i.e. liquid waste transfer is switched from one vessel to another upon detection of a preset level of liquid waste content in the first vessel. The switch mechanism allows heating of one vessel while another vessel is engaged for collecting the liquid waste. This allows to decontaminate liquid waste during the production or purification process without the need to interrupt that process, thereby contributing to the continuous character of the process. Furthermore, liquid waste produced during the production and/or purification process can be decontaminated in the liquid waste vessels without transfer to another unit or isolator. This is referred to as in-line decontamination.

In another embodiment, heating of the liquid waste in the liquid waste vessels occurs in all liquid waste vessels simultaneously during or after completion of the production and/or purification process. In-line decontamination of the liquid waste is advantageous as it accelerates the production and purification process thereby rendering the viral production system more efficient.

In another or further embodiment, the liquid waste vessel is provided with a mixing system that allows mixing of the vessels' liquid content during decontamination. Such a mixing system can, for example, comprise a device that allows shaking of the vessel, or a device that allows stirring of the vessels' content, such as a magnetic stirrer. Mixing of the vessels' liquid content during heating further allows to ensure homogenous heating of the liquid waste inside the vessel is achieved during decontamination. The presence of ‘cold spots’ inside the liquid waste vessel as well as the temperature distribution of the liquid inside the vessel can easily be mapped using methods known to the skilled person such as thermal mapping and mathematical modeling.

The liquid waste vessels can, in another or further embodiment, optionally be provided of pumps fitted on conduits that assist the liquid to flow out of the liquid waste vessel once decontamination has terminated.

Efficient decontamination is calculated based on the log₁₀ reduction of the initial viral titer, which can be different for different parts of the system as the viral titer depends on the progression of the virus purification process. In the case of liquid waste, a distinction in viral titer can be made between the production unit and the purification unit. Depending on the heating temperature decontamination of the liquid waste will take more time (at a lower temperature) or less time (at a higher temperature). The time needed to reduce the titer of a specific virus by one log₁₀ at a certain temperature is known or can otherwise be measured using methods known to the person skilled in the art. Note that this value is dependent on the specific virus used and on the temperature targeted during decontamination. Based on this 1 log₁₀ reduction time, the skilled person can calculate the time needed to decontaminate the system. Preferably the decontamination time is calculated such that less than 10⁻⁶ viral particles/m³ or less than 1×10⁻⁶ TCID₅₀/ml are estimated to remain active in the liquid, this time can then be doubled or tripled to further ensure no active viral particles are present in the liquid waste after decontamination. Once decontaminated, the liquid waste may be transferred outside the units, isolators or containment enclosure and stored outside the latter. Alternatively, the decontaminated liquid waste can be pumped out of the vessel directly into a waste stream located outside the enclosed environment.

During decontamination, whether or not it is achieved using the decontamination of the system via heated air or liquid, or the decontamination of the collected liquid waste, the temperature of the air, liquid and/or the temperature of the liquid waste will be monitored. Hence, in an embodiment, the disclosed biomolecule production system comprises one or more temperature sensors for monitoring the temperature of the heated air or liquid through-out said system during and after the decontamination procedure. Monitoring of the temperature is useful for adjusting the heating. This might be necessary to avoid exceeding the desired temperature unnecessarily and/or to increase the heating when the desired temperature hasn't been reached. These adjustments can be performed manually, but they are preferably performed in an automated fashion. Preferably, monitoring of the temperature during decontamination is performed using temperature sensors, more preferably using multiple sensors throughout the system. Monitoring of the temperature of the air, liquid and/or the temperature of the liquid waste vessel's circulation conduit allows to adjust the heating accordingly to verify that the desired temperature is reached uniformly throughout the whole system and thus contributes to reduce the risk of virus survival in areas where the desired temperature was not reached. In addition, temperature monitoring throughout the system enables the start of decontamination timing to begin only when a uniform steady state temperature is reached. In another or further embodiment, an alert is issued if the desired temperature has not been reached. This further contributes to reducing the risk of release of active viral particles due to virus survival in areas where the desired temperature was not reached for the time period required for efficient viral inactivation.

As described above, liquid waste generated by the system during the production and/or purification process is decontaminated using one or more liquid waste vessels during or after the production and/or purification process.

After termination of the production process contaminated parts of the system for the production of virus particles are exposed to heated air and/or heated liquid at a temperature of at least 60° C., preferably at least 70° C., at least 90° C. thereby inactivating remaining virus particles in said system. Liquid or air used for decontamination is preferably provided in-line to the system. In an embodiment, production and purification of viral particles take place inside one or more isolators present in the system, accordingly decontamination of the system requires decontamination of all materials within those isolators. When these isolators are provided of heating mechanisms and fans and/or pumps according to an embodiment of the disclosure, the isolators have built-in decontamination function. Liquid and/or air which is used for decontamination can be heated before and/or during the decontamination procedure in appropriate vessels, tanks, in situ or in-line.

While the use of heated air to decontaminate the system entails large advantages such as the fact that it does not leave any residues in the system and that it reaches places which are harder to reach using other methods, some parts of the system could potentially benefit more from liquid borne heat as a decontamination agent.

Bioreactors, in particular, have been found to be prone to a heterogeneous temperature distribution when decontamination according to the method described above is performed using heated air. Therefore, and in a further embodiment, decontamination of the bioreactor is preferably performed using heated liquid. The liquid may be heated prior to filling of the bioreactor and/or may be heated inside the bioreactor using the integrated heating device present in the bioreactor.

Once the system (or parts thereof) has been exposed to heat for the predetermined amount of time, the system (or parts thereof) can additionally be exposed to additional decontaminating agents such as for example vaporized hydrogen peroxide (VHP). In particular, VHP is obtained as a solution of hydrogen peroxide in water, and is then vaporized using a vaporizer. The heated vapor or gas can then, in an embodiment, be circulated through the system as an additional decontamination strategy. As hydrogen peroxide readily decomposes to form water and oxygen, VHP possesses none of the environmental risks associated with formaldehyde. The broad spectrum efficacy of VHP has been shown against a wide range of micro-organisms including, in addition to viruses, fungi, bacteria and bacterial spores. Treatment with VHP in addition to exposure to heat further assures decontamination of the system, including in particular, filters such as HEPA filters which are part of the HAVC of the system. In an embodiment, a VHP bottle or unit may be connected to the inlet of the HEPA filters in order to undergo a VHP cycle and decontaminate the HEPA filters.

In an embodiment, one or more VHP decontamination units may be present in the system. In an embodiment, where the units are present in isolators, at least one or more isolators may be provided with a VHP decontamination unit. In an embodiment, every isolator is provided with a VHP decontamination unit. Said decontamination unit may be positioned inside the isolator. In an embodiment, the VHP unit is provided as an external unit, positioned outside the isolators which is connectable to the isolators for the purpose of decontaminating said isolator. Said decontamination unit may be mobile. Said decontamination unit may be sequentially connected to one of the isolators. In another embodiment, every isolator is provided with a decontamination unit.

VHP decontamination of the units or isolators may be simultaneous or sequential. In an embodiment, one or more units or isolators may undergo a VHP decontamination cycle as described above, whereas other units or insulators may be in an active (production) mode. For instance, in an embodiment, the production and purification units/isolators may, once they have terminated their activities, be VHP decontaminated, whereas the downstream inactivation unit or isolator is still active and still performing the inactivation cycle. In another embodiment, all units or isolators will be VHP decontaminated simultaneously.

As the transfer of fluid or gas through the system is provided by conduits, it may be preferable to further clean these conduits after decontamination has been performed. Accordingly, in a further or other embodiment the conduits present in the system can be reversibly removed from the system after heat inactivation of the potentially remaining viral particles has been performed. Preferably, the conduits are removed from the system after decontamination, and are sterilized prior to return to said system. In another or further embodiment, all removable parts of the system are disassembled and either sterilized by autoclaving or discarded in the case of disposable components, after decontamination has been performed. In addition to decontamination, sterilization of the conduits and other removable parts of the system contributes to prevent contamination of the biomolecule production process with bacterial contaminants in a subsequent biomolecule (e.g. virus) production cycle, as this could significantly affect the yield and/or quality of the purified virus. In another or further embodiment, and as already mentioned above, the conduits can only be accessed after decontamination of the system has been recorded to allow opening of the isolators and/or of the containment enclosure comprising the individual isolators. In a further embodiment, the reusable and removable components of the system are cleansed prior to autoclaving to remove non-living contaminants such as, but not limited to, cell debris and media residues.

By preference, the biomolecule production system is able to execute the method for decontamination as described above. The biomolecule production system of the present disclosure allows down-scaling of the infrastructure required for biomolecule production on an industrial level. In addition, high yield of the purified biomolecule is obtained using the biomolecule production system thereby reducing the costs of the final product. This eventually results in a lower investment and production cost, which is a considerable advantage. The major advantage of the currently disclosed system, in addition to ensuring efficient, rapid and cost-effective biomolecule production, is that it assures efficient decontamination of the system, thereby limiting the risk of infection for the user as well as significantly reducing the risk of release of active virus into the environment.

As mentioned earlier, access to the containment enclosure as well as to the isolators can be controlled automatically by a process control device. In addition to collecting, monitoring and/or recording data on actions performed by the components of the system, the process control device can be used to perform the biomolecule production and optionally the biomolecule purification processes. In another or further embodiment, the process control device can be used to perform the decontamination of the system. The steps required for heat decontamination are thereto pre-programmed in the system. In a further embodiment, an operator can easily select the desired decontamination process using an interface of the process control device, based on a set of pre-programmed decontamination procedure available on the device. These pre-programmed decontamination procedures are dependent on several parameters including, but not limited to, the biomolecule production (and purification) system used, the type of biomolecule produced, the temperature of the liquid and/or air used to expose the system during decontamination and the aimed temperature of the liquid waste in the liquid waste container(s) present in the system during decontamination. Alternatively, decontamination of individual parts of the system can be initiated automatically by the process control device upon completion of one or more predetermined tasks. For example, the process control device can initiate decontamination of a first liquid waste vessel present in the purification unit of a biomolecule production system before the end of the production phase and engage a second liquid waste vessel for the collection of the liquid waste originating from the production process.

In a further aspect, the current disclosure provides a biomolecule production system comprising at least one liquid waste vessel with built-in decontamination function as described above. More in particular, the disclosure provides a vessel or container for holding a liquid, comprising a heating device and further comprising or potentially comprising active virus particles, characterized in that the heating device is adapted for inactivating the virus particles and wherein the container is an isolator.

The present disclosure will be now described in more details, referring to figures that are not limitative.

### DETAILED DESCRIPTION OF FIGURES

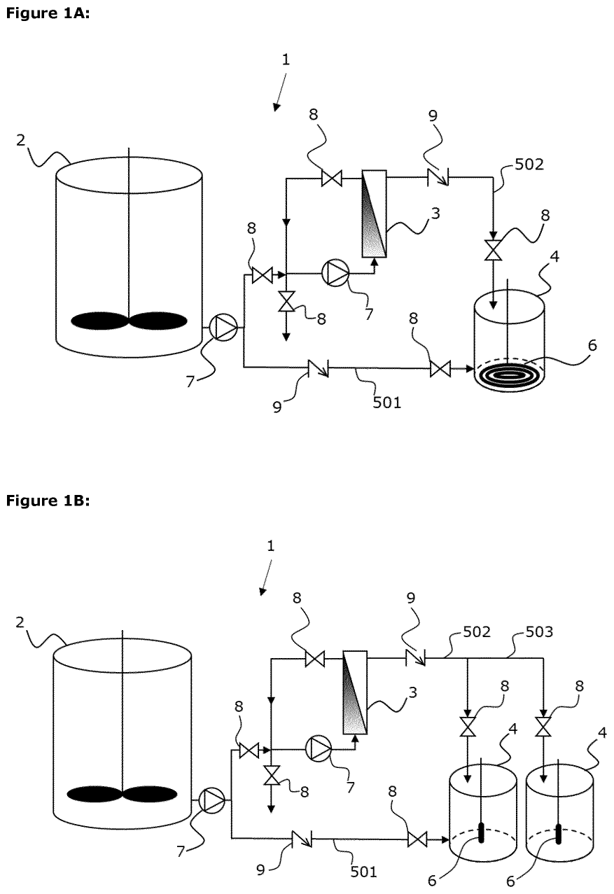

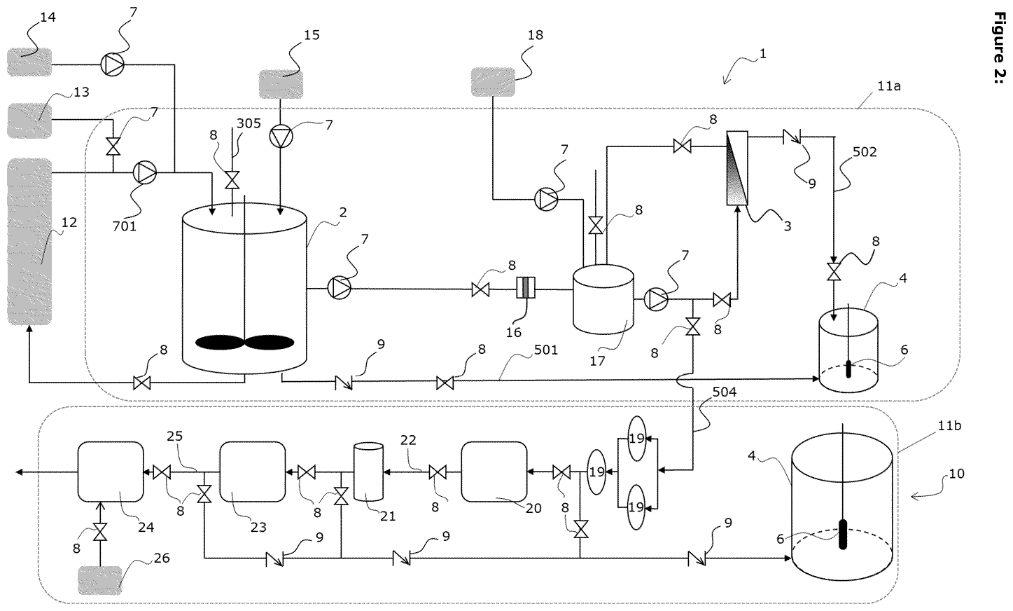

### FIGS. 1A and 1B: Schematic Overview of a Production Unit of a Biomolecule Production System According to Embodiments of the Disclosure

A schematic overview is shown of a production unit (1) of a virus production system comprising a bioreactor (2) including a chamber suitable for having a liquid comprising cells and viral particles, and a concentration device (3). Liquid waste generated in the production unit (1) is collected in one or more liquid waste vessels (4). FIG. 1A shows a production unit (1) with one such liquid waste vessel (4) and FIG. 1B shows a production unit (1) with two such liquid waste vessels (4).

The bioreactor (2) and the concentration device (3) are connected to the liquid waste vessel(s) (4) by a conduit (501) allowing liquid waste transport from the bioreactor (2) to the liquid waste vessel (4) and by a conduit (502) allowing liquid waste transport from the concentration device (3) to the liquid waste vessel (4). Accordingly, liquid waste generated in the production unit (1) is collected in the liquid waste vessel(s) (4). When more than one liquid waste vessel (4) is provided in the system, these liquid waste vessels (4) are fluidly connected to each other by a conduit (503).

The one or more liquid waste vessels (4) are equipped with a heating system or element that allows heating of the liquid waste inside the liquid waste vessel (4) to the desired temperature. Preferably, the liquid waste is heated to a temperature of at least 60° C. for a predetermined amount of time thereby inactivating virus particles potentially present in the liquid waste.

In the embodiments shown in FIGS. 1A and 1B, the liquid waste vessel (4) accommodates an electrical resistor (6) as the heating element. Said heating elements can have any type of suitable configuration such as a tubular configuration, a rod-like configuration, a coiled configuration, a plate configuration. Said heating elements are preferably suited to be immersed in a liquid. Such heating system can alternatively comprise any number of heating elements including other static heating elements such as infrared elements, UV elements, etc. The heating element (6) is submerged in the liquid waste and allows heating of the liquid waste in the liquid waste vessel (4). Preferably, the resistor is positioned near or on the bottom of said vessel, in order to allow optimal heating of its content.

The conduits of the system are fitted with pumps (7) to provide directional liquid flow. In addition, the conduits of the system are provided with valves (8) to control flow distribution. The valves (8) further allow to engage or disengage a specific system segment or conduit. In addition, the conduits (501 and 502) which are connected to the liquid waste vessels (4) are equipped with check valves (9). These check valves (9) prevent backflow of the liquid waste from the liquid waste vessels (4).

The production unit (1) of the virus production system further comprises a heating device for heating air and/or liquid to a temperature of at least 60° C. and one or more pumps for circulating the heated air or liquid through the virus production system or parts thereof, which are not shown on this schematic overview.

### FIG. 2: Schematic Overview of a Biomolecule Production System According to Another Embodiment of the Current Disclosure

The depicted virus production system comprises a virus production unit (1) and a purification unit (10). The production unit (1) is equipped with a bioreactor (2) to produce virus particles and with a concentration device (3) that allows the concentration of viral particles in a cell culture harvest. Preferably, the production unit is contained within a biosafety cabinet or isolator (11a).

A pre-culture of cells suitable for the production of the desired biomolecule is obtained for inoculation in the bioreactor (2). Prior to inoculation, the bioreactor (2) is set up, provided with growth medium from a growth medium tank (12), and the medium is provided into the bioreactor (2) using at least one pump (701). By preference, the medium is pre-heated to a temperature of between 25° C. to 37° C. and mixed prior to transfer to the bioreactor. This ensures that the cells will not perceive a cold-shock when being contacted with new medium (which would negatively affect their growth) as well as ensure that all nutrients in the medium are mixed and present in the required amounts. The medium can be a liquid comprising a well-defined mixture of salts, amino acids, vitamins, carbohydrates, lipids, and one or more protein growth factors. The culture medium serves to deliver nutrients to the cell and conversely, to remove waste products and to prevent a toxic build-up of metabolic waste. The cell culture parameters are also defined prior to inoculation. In the embodiment of FIG. 2, the bioreactor (2) is further connected through conduits with the inoculum vessel (13) comprising the rinsed, detached and neutralized cell preculture in suitable growth medium, and an additives vessel (14) comprising additional additives which are known to a person skilled in the art such as for example growth factors. The bioreactor (2) can further be provided with a gas inlet (not shown) and/or outlet (305) and a base (15) inlet for regulation of the pH inside the bioreactor (2). Note that all buffers and media tanks are located outside the isolator (11a). After inoculation, the cells are grown in the bioreactor (2) for a suitable time or until the desired cell density is achieved. Prior to infection of the cells with the desired virus, the growth medium used is exchanged with growth medium suitable for viral particle production. The discarded growth medium is not infected with viral particles and can therefore be collected in the growth media tank (12) which is located outside the isolator.

During a next phase, viral production takes place. The bioreactor (2) is thereto operated in perfusion mode with in-line concentration. To avoid clogging of the ultrafiltration device present in the concentration device (3), the liquid is first passed through a pre-filter (16) which removes large solid particles from the liquid but is permeable to the biomolecule of interest. Preferably, the pre-filter has a pore size of approximately 125 μm and a cutoff of approximately 100 kDa. Concentration of the liquid is performed by passing the liquid through a concentrator (3) which is an ultrafiltration device, preferably a tangential flow filtration (TFF) device. This concentration device (3) allows to concentrate the virus by discarding the permeate while retaining the retentate comprising the viral particles. The discarded permeate contains mainly liquids and small solutes and potentially also contains remaining viral particles. The permeate discarded by the concentration device (3) is collected in a liquid waste vessel (4), located inside the isolator (11a). The volume of this liquid waste vessel (4) may be between 1 and 1000 L, such as 10 L. The concentration device (3) is thereto connected to the liquid waste vessel(s) (4) by a conduit (502) allowing liquid waste transport from the concentration device (3) to the liquid waste vessel (4). The concentration device (3) allows the volume of liquid comprising the virus to be drastically reduced prior to further purification.

The liquid waste vessel (4) of the purification unit is equipped with a heating system allowing heating of the liquid waste to a temperature of at least 60° C., preferably to a temperature of at least 70° C., thereby inactivating virus particles which might be present in the liquid waste. In the current embodiment, a heating element in the form of an electrical resistor (6) is present in the liquid waste vessel (4). Alternatively, heating of the liquid waste vessel could be achieved by providing it with a heating jacket at its circumference, allowing heating of the liquid to a desired temperature. Yet another alternative would be to include a circulation conduit, allowing circulation of liquid waste from and to said vessel, and wherein said conduit comprises a heating device such as an electrical resistor for heating said liquid waste during circulation. The volume of the liquid waste vessel (4) may be between 1 and 100 L, for instance 70 L. While the resistors of the liquid waste vessels in FIG. 2 are presented as rods, it will be obvious to the skilled person that such resistors could equally be provided in the form of a wire coil, as shown on FIG. 1, or other suitable shape and size.

The virus production system of the current disclosure makes use of pumps (7, 701) and valves (8), which are fitted on the conduits of the system, to induce directional flow of the liquid through the system and to allow reversible engaging and disengaging of different parts of the system. Additionally, the system's conduits allowing liquid waste transport to the liquid waste vessel (4) are, in the depicted embodiment, equipped with check valves (9) that prevent backflow of the waste liquid from the liquid waste vessel.

After peaking of the viral production phase, the retentate is harvested in the harvest vessel (17). The bioreactor (2) is rinsed with clean medium once it is empty. The remaining liquid is subsequently recirculated through the concentration device (3) until the desired volume reduction is achieved. This generates an additional large volume of contaminated liquid waste which is collected in the liquid waste vessel (4). Finally, the recirculated output of the concentration device (3) is harvested in the harvest vessel (17) thereby obtaining a concentrated cell culture harvest. Alternatively, the retentate, which is recirculated between the concentrator (2) and the bioreactor (2) in the absence of a harvest vessel (17) is harvested by collecting it in the bioreactor (2).

Optionally, the pH of the concentrated cell culture harvest is adjusted to the desired value for downstream purification steps using a pH adjustment solution (18) which is connected to the harvest vessel (17) as shown in FIG. 2 or to the bioreactor (2). In addition, an optional endonuclease treatment can be performed on the concentrated cell culture harvest to degrade DNA and RNA present in the concentrated cell culture harvest while leaving proteins intact. An endonuclease treatment step can contribute to the prevention of aggregation in the concentrated cell culture harvest, thus providing optimal conditions for further purification steps.

Further, purification of the virus preferably takes place in a second isolator (11b) comprising the purification unit (10). The production unit (1) and the purification unit (10) are fluidly connected to each other through a conduit (504) suitable for liquid transfer.

The purification unit (10) according to the current embodiment is equipped with a clarification device, a chromatography device, a virus inactivation device and a liquid waste vessel (4). The clarification device comprises a number of anion exchange depth filters (19). The clarification device removes residual solid contaminants from the product stream assuring the correct functioning of the subsequent purification steps. The clarified cell culture harvest or clarified retentate is collected in a clarified harvest vessel (20) prior to transfer to the system's chromatography device. The chromatography device allows further purification of the virus. The chromatography device according to the current embodiment comprises a mixed mode chromatography column (21) which is suited for continuous mode operation. The chromatography device and the clarification device are connected by a conduit (22) facilitating liquid transport from the clarification device to the chromatography device. After chromatography has been performed, the purified virus is temporarily stored in a chromatography harvest vessel (23) wherein the conditions (pH, salt concentration) of the liquid can be adjusted. Finally, the purified virus is transferred to a virus inactivation device which is suited for final inactivation of the purified virus and formulation thereof. This virus inactivation device comprises an inactivation vessel (24) to which formaldehyde buffer can be administered from a formaldehyde buffer tank (26) located inside the isolator (11b). The chromatography harvest vessel (23) and the inactivation vessel (24) are connected by a conduit (25) facilitating liquid transport from the chromatography harvest vessel (23) to the inactivation vessel (24).

Liquid waste is generated in the purification unit, for example, by priming of the clarification device, sanitization/equilibration/washing/elution of the chromatography column and final formulation and inactivation of the purified virus. This liquid waste is collected in the purification unit's liquid waste vessel (4), which is located inside the respective isolator (11b). This liquid waste is collected in a liquid waste vessel (4), which is included in the purification unit. Decontamination of the liquid waste in the liquid waste vessel (4) in the purification unit can be performed as described for the liquid waste vessel (4) in the production unit. The liquid waste vessels (4) of both units are not necessarily of the same volume. Often a larger volume of liquid waste is generated during the purification process, therefore requiring a liquid waste vessel (4) with a larger volume or an additional liquid waste vessel within the same unit.

Once the viral production has finalized, the system is decontaminated by pushing heated air or liquid through the system or parts thereof. The system thereto comprises a heating device for heating air and/or liquid to a temperature of at least 60° C., preferably at least 70° C., at least 80° C., at least 90° C. The heated air or liquid is circulated through the production and purification unit and through the devices present in said units. This is achieved by pumping the heated air or liquid through the conduits connecting the devices in said units and allowing passage of the heated air and liquid through the devices.

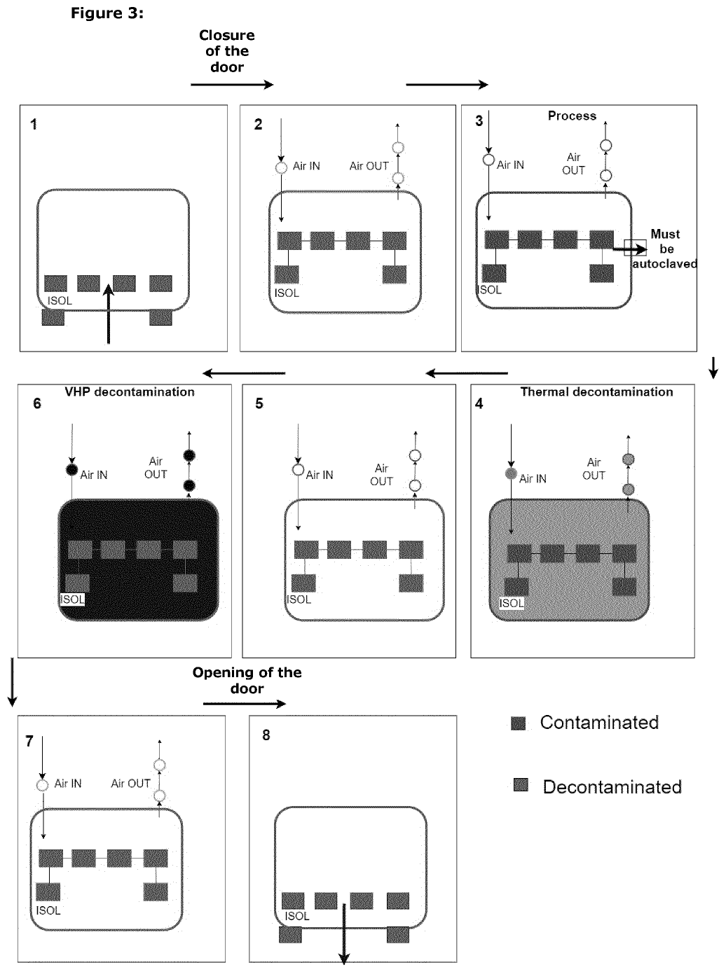

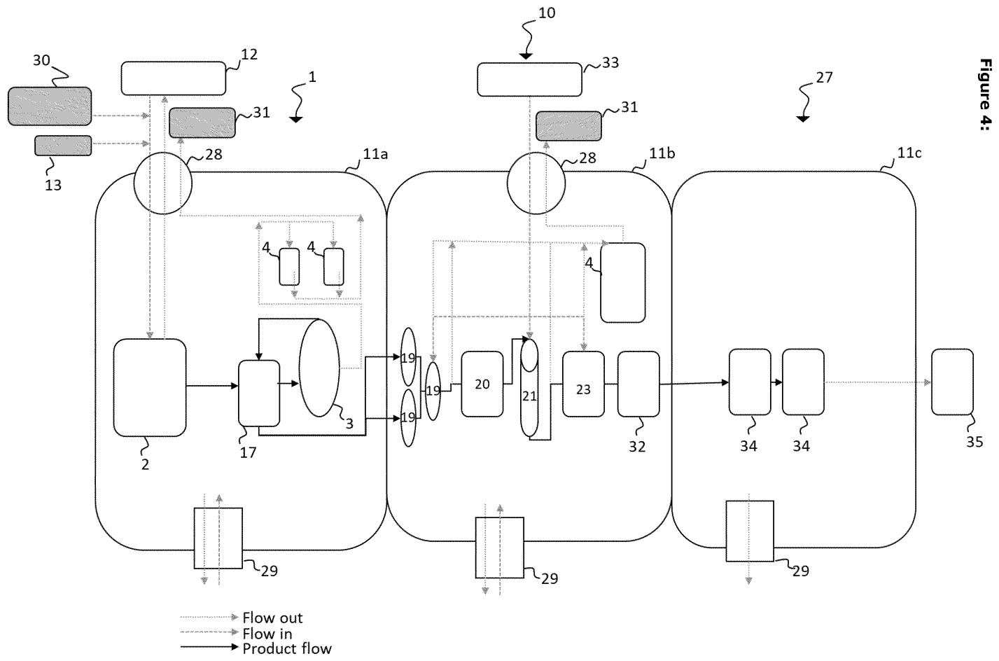

### FIG. 3: Decontamination of a Biomolecule Production System According to an Embodiment of the Disclosure

A schematic overview is shown of the decontamination of a biomolecule production system according to the disclosure. The system is represented by an isolator (ISOL) wherein equipment for the production of the biomolecule is present (including a bioreactor) which are interconnected by conduits. The air flowing into the isolator is pre-filtered using a HEPA filter and the air flowing out of the isolator is filtered through two series of HEPA filters.

The (de)contamination status of the system's components is shown before, during and after the biomolecule production process. Before the process, every component is considered as non-contaminated. Unfolded conduits enter the isolator through the opened entrance (Panel 1). The entrance is then closed and the conduits are folded inside the isolator by the operator (Panel 2).

During the production process the ambiance of the isolator (ISOL) and all the components inside the isolator (ISOL) are both considered as contaminated as well as the HEPA filters located in the air flow out and in. Contaminated material can go out the isolator through the rapid transfer port/rapid transfer container (RTP/RTC) system as it is isolated in the RTC and will be decontaminated by autoclave (Panel 3).

After the production (and purification) process, a thermal decontamination cycle is undergone (Panel 4) according to the present disclosure. The system is exposed to heat, preferably at a temperature above 70° C., more preferably above 80° C., or above 90° C. for a predetermined amount of time in order to decontaminate the items inside the isolator (ISOL) as well as the ambiance of the isolator (ISOL) (Panel 5).

Optionally, the system can additionally be exposed to a vaporized hydrogen peroxide (VHP) fumigation cycle to further ensure decontamination of the three HEPA filters (Panel 6). VHP decontamination may occur by means of a mobile VHP unit, which is either part of one of more isolators, or which is positioned outside the isolators and connectable to said isolators.

At the all the components of the system are decontaminated (Panel 7) and the entrance, which is also an exit, of the isolator (ISOL) can be opened (Panel 8) and components can optionally be removed from the isolator (ISOL).

### FIG. 4: Schematic Overview of Liquid Waste Flow in a Biomolecule Production System According to an Embodiment of the Disclosure

The depicted biomolecule production system comprises a virus production unit (1), a purification unit (10) and an inactivation unit (27). Each unit is located in a separate isolator (11a, 11b, 11c). Arrows indicate the different liquid flow streams including the liquid flow from outside to inside the isolators (dashed line), the liquid flow from inside to outside an isolator (dotted line) and the product flow through the production and purification system (solid line).

Note that all buffers and media tanks are located outside the isolators (11a, 11b, 11c). The production unit (1) and the purification unit (10) are both provided with a liquid transfer port (28) for aseptic transfer of liquids across the wall of the respective isolator. This liquid transfer port (28) is safe for use in transfer of liquid to highly contaminated environments. In addition, each isolator (11a, 11b, 11c) is provided of a rapid transfer port/rapid transfer container (RTP/RTC) system (29) to allow safe transport of liquids and/or solids into and out of the isolators (11a, 11b and 11c).

The production unit (1) is equipped with a bioreactor (2) to produce virus particles, with a concentration device (3) that allows the concentration of viral particles in a cell culture harvest, with a harvest vessel (17) wherein the concentrated cell culture harvest is collected and with two liquid waste vessels (4) for collecting liquid waste comprising or potentially comprising active virus particles. The liquid waste vessels (4) are equipped to heat the liquid waste.

Liquid flow from outside to inside (dashed line) the isolator (11a) of the production unit (1) according to this simplified embodiment shown in FIG. 4 include growth medium from a growth medium tank (12), inoculum (cells) from the inoculum vessel (13) and medium suitable for viral particle production from the infection media tank (30). It is clear that additional liquid flow from outside to inside (dashed line) the isolator (11a) of the production unit (1) is also possible, as illustrated for example in FIG. 2.

Prior to infection of the cells with the desired virus, the growth medium used is exchanged with growth infection medium from the infection media tank (30). The discarded growth medium is not infected with viral particles and can therefore be collected in the growth media tank (12) which is located outside the isolator (11a). The discarded growth medium is therefore transported through the liquid transfer port (28) from inside to outside the isolator (11a).

In order to infect the cells with the desired virus, the virus is introduced from outside to inside the isolator (11a) through the RTP/RTC system (29). From that point on, the liquid waste which is generated by the production unit (1) is considered to be contaminated with active virus and is collected in a liquid waste vessel (4) which is present inside the isolator (11a) and which is equipped to heat said liquid waste to a temperature of at least 60° C., preferably to a temperature of at least 70° C., thereby inactivating virus particles which might be present in the liquid waste. In the embodiment shown in FIG. 4, two liquid waste vessels (4) which are fluidly connected to each other by conduits are present. However, to be clear one or more vessels (4) can be used.

The embodiment shown in FIG. 4 employs a sequential use of the liquid waste vessels (4). A switch mechanism which might comprise valves may thereto be present on the respective conduits (not shown on FIG. 4) to allow switching liquid waste transfer from one vessel to another. Preferably, switching is performed on an automated fashion based on the measurement of liquid level inside the respective liquid waste vessel (4) using one or more level sensors which are provided inside the vessel (4). The switch mechanism allows heating of one vessel (4) while another vessel (4) is engaged for collecting the liquid waste. This allows to decontaminate liquid waste during the production process without the need to interrupt that process, thereby contributing to the continuous character of the process. Furthermore, liquid waste produced during the production process can be decontaminated in-line in the liquid waste vessels (4). This switch mechanism is especially advantageous when decontamination of large liquid waste volumes is required.

Once decontaminated, the liquid waste is transferred through the liquid transfer port (28) from inside to outside the isolator (11a) where it can be stored in one or more waste collection vessels (31) as shown in the embodiment of FIG. 4. Alternatively, the decontaminated liquid waste can discarded directly into a waste stream located, for example, outside the enclosed environment.

Further purification of the virus takes place in a second isolator (11b) comprising the purification unit (10). The production unit (1) and the purification unit (10) are fluidly connected to each other through a conduit facilitating the product flow. The purification unit (10) according to the current embodiment is equipped with a clarification device and a chromatography device.

The clarification device comprises a number of anion exchange depth filters (19). The clarified cell culture harvest or clarified retentate is collected in a clarified harvest vessel (20) prior to transfer to the system's chromatography device comprising a mixed mode chromatography column (21). The product flow from the clarification device to the chromatography device is facilitated by a conduit which fluidly connects the both devices. The purified virus product obtained after chromatography is temporarily stored in a chromatography harvest vessel (23) prior to transfer to a pre-inactivation vessel (32). In the pre-inactivation vessel (32) addition of an inactivating agent such as formaldehyde to the purified viral product takes place before the product is transferred to one or more inactivation vessels (34) of the inactivation unit (27).

Liquid flow across the isolator's wall is accomplished by a liquid transfer port (28) and/or a RTP/RTC system located on the isolator's (11b) wall. Liquid flow from outside to inside the isolator (11b) of the purification unit (10) includes, downstream processing (DSP) buffers (33) required for the purification process, and which are located outside the isolator (11b). Additionally, the inactivating agent in the currently depicted embodiment is provided from outside to inside the isolator (11b) through the RTP/RTC (29) system.

Liquid waste is generated in the purification unit (10), for example, by priming of the clarification device, sanitization/equilibration/washing/elution of the chromatography column (21). This liquid waste is collected in the purification unit's (10) liquid waste vessel (4), which is located inside the respective isolator (11b). Decontamination of the liquid waste in the liquid waste vessel (4) in the purification unit (10) can be performed as described for the liquid waste vessels (4) in the production unit (1). The liquid waste vessels (4) of both units are not necessarily of the same volume. Often a larger volume of liquid waste is generated during the purification process, therefore requiring a liquid waste vessel (4) with a larger volume or an additional liquid waste vessel within the same unit. Once decontaminated, the liquid waste is transported from the liquid waste vessel (4) to a waste collection vessel (31) located outside the isolator (11b) through the liquid transfer port (28). Alternatively, the decontaminated liquid waste can be discarded directly into a waste stream located, for example, outside the enclosed environment.

Final inactivation and formulation of the purified viral product is performed in the depicted embodiment in the inactivation unit (27) and comprises in the current embodiment two inactivation vessels (34). These inactivation vessels (34) are located inside a third isolator (11c) and are fluidly connected to each other and to the pre-inactivation vessel (32) of the purification unit's (10) isolator (11b) through conduits which facilitate product flow from the purification unit (10) to the inactivation unit (27).

Inactivation of the purified viral product in the system shown in FIG. 4 does not generate liquid waste. Accordingly, no liquid waste vessel is required in the third isolator (11c).

After inactivation and formulation, the inactivated viral product is collected in a final product container (35) located outside the isolator (11c) of the inactivation unit. During the production and the purification process, samples can safely be taken out of the respective isolators (11a, 11b, and 11c) through the RTP/RTC system (29) present on each isolator.

Once the viral production has finalized, the system can be decontaminated according to an embodiment of the method for decontamination of the disclosure, see for example FIG. 3.

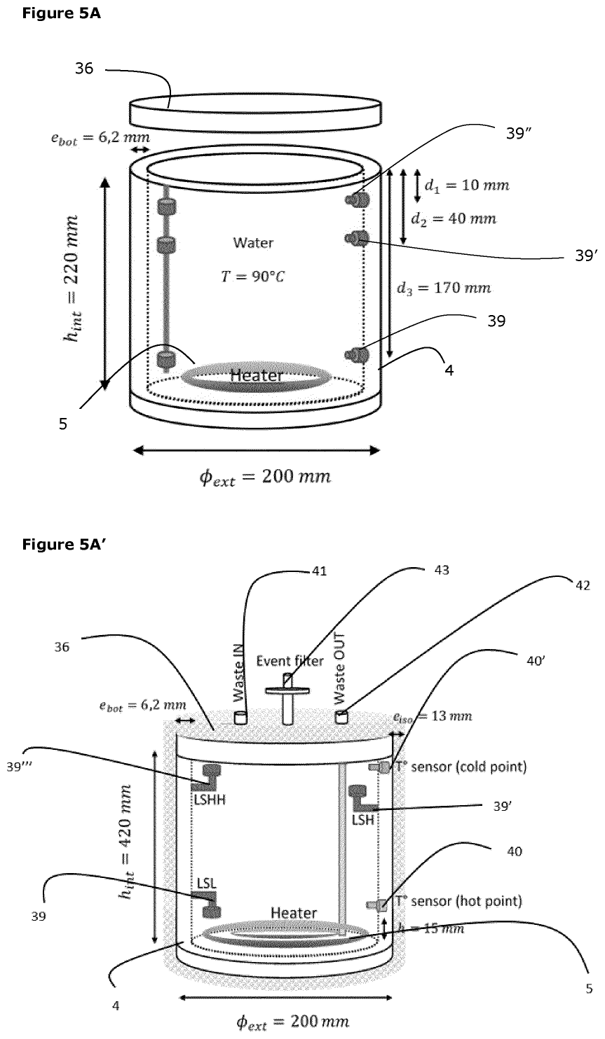

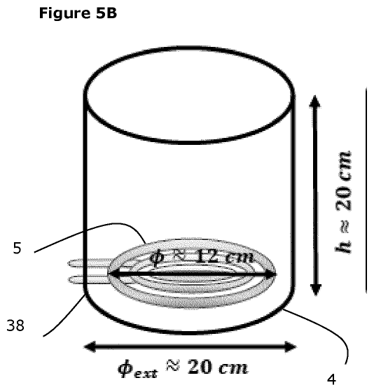

### FIGS. 5A, 5A′ and 5B: Liquid Waste Vessel

FIGS. 5A, 5A′ and 5B show a liquid waste vessel to be used in the biomolecule production system as described above, which allows collecting and decontamination of liquid waste produced by the system. The vessel (4) is a closed vessel, provided by a lid (36), which may be removable. By preference, the vessel is cylindrical or parallelepiped of shape and can have a volume of between 1 and 100 L. The vessel (4) could be produced of any kind of material, known in the art. Preferably a plastic such as a polypropylene is used. Insulation may be provided on the surface of the bottle to limit heat losses during decontamination. A suitable type of insulation is an auto-adhesive sheet wrapping the surface (e.g. Armaflex).

On the bottom of the vessel (4) or at a height above the bottom of the vessel (4) (e.g. 10 to 30 cm from the bottom), a wire coil resistor (5) is positioned which allows heating of the liquid inside the vessel. The position at the lower part of the vessel allows optimal temperature homogeneity. The resistor (5) is designed such that it allows heating of liquid present in the vessel (4), going from room temperature or less to 90° C. or more. In order to fixate the resistor (5) to the vessel (4), the resistor may be connected to the lateral internal surface of the vessel by means of a connection piece (38). The connection will be designed such that it is non-heating.

In the embodiment shown in FIG. 5A, level sensors (39, 39′, 39″) are present in the inside of the vessel (4), positioned at the inner wall of said vessel. At least three levels are present:
a lower level sensor (39) able to detect the fluid immergence of the resistor. Said lower level sensor is placed at or near the level of the resistor in the vessel, being near the bottom or at the lower level of the tank;
one or more level sensors (39′) at a predefined height, which corresponds to a certain volume in the tank (e.g. 5 L of volume)
a safety level sensor (39″) at the top of the tank for safety reasons, to signal when the tank is completely full.

The level sensors as shown on FIGS. 5A and 5A′ are side-mounted single float switch sensors that work as a magnetic switch that activates when the liquid lifts the float. They act as a simple on/off switch a signal easy to handle in our case and doesn't need power. It can be configured as normally open (NO) or normally closed (NC), or depending the direction it is fixed. In another embodiment (not shown) a single multi-float sensor is used.

In an alternative embodiment, shown in FIG. 5A′, both level sensors (39, 39′, 39″) and temperature sensors (40, 40′) are present inside the vessel, positioned at the inner wall of said vessel. In the embodiment shown, at least two temperature sensors are present:
a lower T sensor (40) near the resistor, able to measure the heating of the liquid near the resistor (hot point);
one or more T sensors (40′) at a predefined height, optionally corresponding to a certain volume in the tank (e.g. 5 L of volume), to measure the temperature in the vessel above the resistor (cold point)

The liquid waste vessel (4) is further provided with one or more conduits (41, 42) (shown for instance on FIG. 1A), allowing entrance of the liquid in the vessel (41) as well as evacuation once decontaminated (42). Entering and evacuation may occur via the same conduit, or alternatively separate conduits could be provided. An example of such configuration is shown in FIG. 5A′. FIG. 5A′ equally shows the presence of a vent filter (43) on the lid of the vessel (4). The vessel is vented to compensate the produced vapors.

Two or more liquid waste vessels as shown in FIGS. 5A and 5A′ may be fluidly connected to each other.

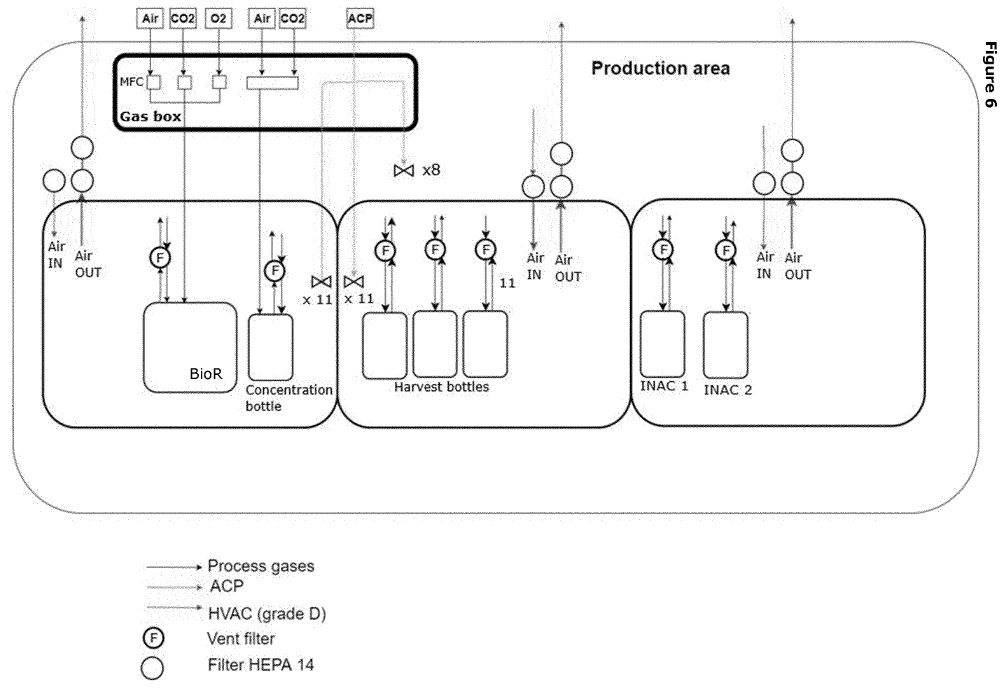

### FIG. 6 Showing an Embodiment of the Gas Flow According to an Embodiment of the System

FIG. 6 shows the gas flow according to an embodiment of the system comprising three isolators: a production isolator comprising a production unit, a purification isolator comprising a purification unit and an inactivation isolator comprising an inactivation unit. In the production isolator, process gases are needed to control critical parameters in both the bioreactor and the intermediate concentration vessel. The flow of CO2, air and O2 is regulated by three mass flow controllers (MFC), located in the gas box, to keep a dissolved oxygen (DO) and a pH constant in the bioreactor. The total flow of gas is constant. In the intermediate concentration vessel, there is no need for O2, because the DO does not need to be regulated. The flow of CO2 and air is then regulated by one MFC in order to keep a pH constant. In the three isolators, a flow in and a flow out of gas go out the bottles to the isolator through the vent filters.

In order to control pneumatic valves, ACP is injected in the gas box in which it is divided in three lines:
The first line directs to pneumatic valves (11 in FIG. 6) located in the production isolator (USP)
The second line directs to pneumatic valves (11 in FIG. 6) located in the purification isolator (DSP)
The third line directs to the DSP buffers to control the pneumatic buffer valves (8 in FIG. 6)

In order to keep a grade D, required for a GMP production, in each isolator, an air flow in and an air flow out is present. The flow in comes from the production area and directs to the inside of the isolator through a HEPA filter. The flow out comes from the isolator and directs to the technical area through two series HEPA filters.

It is supposed that the present invention is not restricted to any form of realization described previously and that some modifications can be added to the presented example of fabrication without reappraisal of the appended claims. For example, the present invention has been described referring to poliovirus, but it is clear that the invention can be applied to Rotavirus for instance or to Influenza virus.

1. A method for decontaminating a biomolecule production system, wherein biomolecules are produced in at least one bioreactor and purified from liquids comprising virus particles thereby generating liquid waste, said method comprising the step of exposing said system or a part thereof to heated air and/or liquid at a temperature of at least 60° C., thereby eliminating or deactivating remaining virus particles in said system.

2. The method according to the previous claim, wherein said air and/or liquid has a temperature of at least 70° C., at least 90° c.

3. The method according to any one of the previous claims, wherein said air and/or liquid has a temperature of at most 99° C.

4. The method according to any one of the previous claims, wherein said heated air and/or a liquid is circulated through said system or a part thereof.

5. The method according to claim 4, wherein said heated air and/or liquid is circulated through conduits of said system or of a part thereof.

6. The method according to any one of claims 4 to 5, wherein said heated air and/or liquid is circulated through said bioreactor after biomolecule production or when part of the biomolecule production steps were executed.

7. The method according to any of the claims 4 to 6, wherein said heated air and/or liquid is circulated through one or more concentration and/or purification devices located downstream from said bioreactor, after biomolecule concentration and/or purification took place in said system.

8. The method according to any one of the previous claims, wherein said air and/or liquid is heated using a heating element, preferably an electrical resistor.

9. The method according to any one of the previous claims, wherein said liquid waste from said system is collected in at least one liquid waste vessel equipped to heat said liquid waste.

10. The method according to previous claim 9, wherein said system comprises one or more concentration devices, for concentrating a biomolecule harvest from said bioreactor, and wherein liquid waste from said concentration devices is collected in one or more liquid waste vessels equipped to heat said liquid waste.

11. The method according to any of the claim 9 or 10, wherein said system comprises one or more purification devices downstream from said bioreactor, and wherein liquid waste from said purification devices is collected in one or more liquid waste vessels equipped to heat said liquid waste.

12. The method according to any of claims 9 to 11, wherein said liquid waste vessel is heated.

13. The method according to any of claims 9 to 11, wherein said liquid waste vessel comprises a circulation conduit, wherein said liquid waste is circulated from and to said vessel, and wherein said conduit is heated during circulation, preferably to a temperature of at least 60° C.

14. The method according to any of the previous claims, wherein the temperature of said air, liquid and/or circulation conduit is monitored during decontamination.

15. The method according to claim 14, wherein said monitoring occurs via temperature sensors.

16. The method according to claim 14 or 15, wherein an alert will be issued if the desired temperature has not been reached.

17. The method according to any one of the previous claims, wherein said system or a part thereof is exposed to infrared or ultraviolet radiation.

18. The method according to claim 17, wherein sources of infrared or ultraviolet radiation are positioned at distinct locations of said system.

19. The method according to claim 18, wherein sources of infrared radiation are positioned at distinct locations of said system, thereby generating a temperature of at least 60° C.

20. The method according to any of the previous claims, wherein said system or parts thereof is decontaminated via VHP.

21. The method according to claim 20, wherein said VHP decontamination occurs by means of a mobile VHP unit.

22. The method according to any of the previous claim 20 or 21, wherein said VHP decontamination is sequential or simultaneous throughout various compartments of said system.

23. The method according to any of the claims 20 to 22, wherein VHP decontamination may take place in one part of said system, while a second part of said system is still active.

24. A biomolecule production system comprising at least a production unit comprising a bioreactor, characterized in that said system further comprises a heating element for heating air and/or liquid to a temperature of at least 60° C. and one or more pumps for circulating the heated air and/or liquid through the biomolecule production system or parts thereof.

25. Biomolecule production system according to claim 24, characterized in that said system further comprises a purification unit comprising one or more biomolecule purification devices.

26. Biomolecule production system according to any one of claims 24 to 25, further comprising one or more liquid waste vessels suitable for receiving liquid waste generated from said production unit and/or purification unit, wherein said liquid waste vessels are equipped to heat said liquid waste.

27. Biomolecule production system according to any one of claims 24 to 26, wherein said production unit comprises one or more concentration devices, for concentrating a harvest from said bioreactor, and wherein one or more liquid waste vessels are present in said production unit, for collecting liquid waste from said concentration devices, said liquid waste vessels are equipped to heat said liquid waste.

28. Biomolecule production system according to any one of claims 25 to 27, wherein said purification unit comprises one or more purification devices, and wherein one or more liquid waste vessels are present in said purification unit, for collecting liquid waste from said purification devices, said liquid waste vessels are equipped to heat said liquid waste.

29. Biomolecule production system according to any one of claims 26 to 28, wherein said liquid waste vessels comprise a circulation conduit, allowing circulation of liquid waste from and to said vessel, and wherein said conduit comprises a heating element for heating said liquid waste during circulation.

30. Biomolecule production system according to claim 29, wherein said heating element of said conduit is an electrical resistor.

31. Biomolecule production system according to any one of claims 24 to 30, wherein one or more units comprise at least two liquid waste vessels, which are fluidly connected to each other.

32. Biomolecule production system according to any one of claims 24 to 31 wherein said system comprises one or more temperature sensors for controlling the temperature of the heated air or liquid when circulating through said system.

33. Biomolecule production system according to any one of claims 24 to 32, wherein said system comprises one or more sources of infrared and/or ultraviolet radiation and wherein said sources are positioned on distinct locations of said system.

34. Biomolecule production system according to any one of the preceding claims, wherein said system is located in a containment enclosure.

35. Biomolecule production system according to claim 34, wherein outside access to said containment enclosure is prohibited until decontamination of the system has been recorded.

36. Biomolecule production system according to any one of the preceding claims, wherein said units are comprised in separated isolators which are connected to or separated from one another by partitions wherein the configuration of said partitions can be brought in an open or closed configuration.

37. Biomolecule production system according to any one of the preceding claims, wherein said biomolecule is a virus.

38. Biomolecule production system according to any of the previous claims, wherein said system comprises a VHP decontamination unit for VHP decontamination of said system or parts or surfaces thereof.

39. Biomolecule production system according to claim 38, wherein said VHP decontamination unit is mobile and connectable to an isolator.

40. Biomolecule production system according to claim 38 or 39, wherein each isolator is connected to a VHP decontamination unit.

41. Biomolecule production system according to any of the previous claims 36 to 40, wherein at least some of the walls of said isolator are insulated, preferably by a material chosen from glass wool, fiber glass and/or neoprene.

42. Use of a biomolecule production system according to any one of claims 24 to 41 for the production of viruses and/or viral vaccines.

43. Use of a biomolecule production system according to claim 42 wherein said viral vaccine is an inactivated polio virus vaccine.

44. A liquid waste vessel, which is closable from the outside environment and wherein the vessel is equipped for receiving liquid waste generated by a biomolecule production system, wherein said liquid waste vessel is further equipped with a heating element, preferably a wire resistor, positioned at the bottom or lower level of said vessel to heat said liquid waste and one or more level detectors present inside the vessel to detect the level of liquid in said vessel.

45. Liquid waste vessel according to claim 44, wherein said waste vessel is equipped with at least three level sensors, positioned on distinct positions on the inner wall of said vessel.

46. Liquid waste vessel according to any of the previous claims, further comprising one or more temperature sensors, positioned on distinct positions on the inner wall of said vessel.

47. A biomolecule production system comprising the liquid waste vessel of any of the claims 44 to 46.

48. The biomolecule production system according to claim 48, wherein said production unit comprises one or more concentration devices, for concentrating a harvest from said bioreactor, and wherein one or more of the liquid waste vessels are present in said production unit, for collecting liquid waste from said concentration devices.

49. The biomolecule production system according to any one of claims 47 to 48, wherein said purification unit comprises one or more purification devices, and wherein the one or more liquid waste vessels are present in said purification unit, for collecting liquid waste from said purification devices.

50. The biomolecule production system according to any one of the previous claims wherein said one or more liquid waste vessels comprise a circulation conduit, allowing circulation of liquid waste from and to said vessel, and wherein said conduit comprises a heating device for heating said liquid waste during circulation.

51. The biomolecule production system according to any one of the previous claims wherein one or more units comprise at least two liquid waste vessels, which are fluidly connected to each other.

52. The biomolecule production system according to any one of the preceding claims, wherein said system is located in a containment enclosure.

53. The biomolecule production system according to any one of the preceding claims, wherein said system is located in an isolator.

54. The biomolecule production system according to any one of the preceding claims, wherein said system is located in one or more isolators.

55. The biomolecule production system according to claim 53, wherein at least one liquid waste vessel is located in each of the one or more containment enclosures.

56. A method for decontaminating a biomolecule production system, wherein biomolecules are produced in at least one bioreactor and purified from liquids comprising virus particles thereby generating liquid waste, said method comprising circulating heated air at a temperature of at least 70° C. through said system and parts thereof and collecting liquid waste generated during the production in one or more liquid waste vessels present in said system, said liquid waste inside said waste vessel is heated by means of a heating element positioned at the bottom or lower level of said vessel wherein the level of liquid waste in said vessel is monitored by one or more level detectors present inside said vessel.
